(19) United States
(12) Patent Application Publication
(10) Pub. No.: US 2022/0220481 A1
(43) Pub. Date: Jul. 14, 2022

(54) LIPID OLIGONUCLEOTIDE ANTISENSE AGAINST ANTIBIOTIC RESISTANCE

(71) Applicants: Institut National de la Santé et de la Recherche Médicale (INSERM), Paris (FR); Centre National de la Recherche Scientifique, Paris (FR); Université de Bordeaux, Bordeaux (FR)

(72) Inventors: Tina KAUSS, Bordeaux Cedex (FR); Corinne ARPIN, Bordeaux Cedex (FR); Philippe BARTHELEMY, Bordeaux Cedex (FR); Phuoc Vinh NGUYEN, Tours (FR)

(21) Appl. No.: 17/609,591

(22) PCT Filed: May 7, 2020
(86) PCT No.: PCT/EP2020/062730

§ 371 Date: Nov. 8, 2021

(30) Foreign Application Priority Data
May 9, 2019 (EP) 19305585.2

(51) Int. Cl. C12N 15/113; A61K 31/545; A61K 31/427; A61K 31/7105; A61P 31/04

(52) U.S. Cl. CPC C12N 15/1137; A61K 31/545; C12N 2310/11; A61K 31/7105; A61P 31/04; A61K 31/427

(57) ABSTRACT

The present invention relates to the treatment of infections due to antibiotic-resistant bacteria. Antimicrobial resistance (AMR) has been observed at dangerously high levels worldwide and alternative strategies are urgently needed. Antisense therapy has been identified as potential therapeutic tool for tackling AMR. However, in the context of AMR, since the antisense oligonucleotides have to reach the target mRNA to be efficient, the cellular uptake inside prokaryotic cells is a critical issue. The inventors demonstrated that antisense oligonucleotide sequences, in particular targeting the bla_{CTX/M15} gene, featuring a lipid moiety conjugated to the ASO extremity show a particularly efficient intracellular penetration in prokaryotic cells and that these lipid-modified antisense oligonucleotides can show a further improved enzymatic stability with phosphorothioate chemistry (PTO). In particular, the present invention relates to an antisense oligonucleotide modified by substitution at the 5′ or the 3′ end by a lipid moiety, wherein said antisense oligonucleotide specifically targets an mRNA encoding a CTX-M extended spectrum β-lactamase. Another object of the invention concerns the antisense oligonucleotide of the invention for use for treating a bacterial infection, in particular due to bacteria resistant to 3^{rd} generation cephalosporins.

The present invention concerns the treatment of bacterial infections while avoiding resistance of these bacteria to this antibacterial treatment.

Since their discovery, antibiotics have revolutionized the medical treatments of patients with bacterial infections by saving numerous lives. They represent a major therapeutic medical tool, which can be used in many treatments, including infections, chemotherapies, transplantation, and surgery for example.

However, antimicrobial resistance (AMR) has been observed at dangerously high levels worldwide (Spellberg et al. (2013) Engl. J. Med. 368:299-302) and alternative therapeutic strategies are urgently needed. Among the different resistance phenomena, the AMR involving the broad-spectrum cephalosporins, including third-generation (3CGs), one of the major class of antibiotic used worldwide, has become a major public health issue (Rossolini et al. (2008) Clin. Microbiol. Infect. 14(suppl 1):33-41). For this β-lactam family, the main resistance mechanism in enterobacteria, is characterized by the production of Extended-Spectrum β-lactamases (ESBLs) with the most widespread type of ESBL in European countries, CTX-M-15 (Bevan et al. (2017) J. Antimicrob. 72:2145-2155).

Different approaches have been developed to address AMR, including the improvement of intracellular delivery of the antibiotics (Abed et al. (2015) Sci. Rep. 5:13500), the use of natural lipopeptide antibiotic tripropeptin C, or β-lactamases inhibitors, for example. However, in the case of small drug inhibitors for example, inhibitor-resistant β-lactamases (IRTs) have developed over time, indicating that new approaches must be explored.

Recently, antisense therapy has been identified as potential therapeutic tool for tackling AMR. Antisense oligonucleotides (ASO) hybridize with mRNA, which inhibit the expression of the gene responsible of the resistance via different possible mechanisms. In this context, ASO represent a promising strategy to restore the resistant bacteria sensitivity to current antibiotics treatments, in particular 3GCs (Readman et al. (2016) Front Microbiol. 7:373; Meng et al. (2015) J. Antibiot. (Tokyo) 68:158-164). However, despite their high potential, the cellular uptake of oligonucleotides remains one of the key steps for eliciting their biological activity, as the targeted mRNAs are located inside the cells.

Recently, the inventors demonstrated that Lipid-oligonucleotide conjugates improve cellular uptake and efficiency of antisense in eukaryotic prostate cancer cells (See International application WO2014/195432). However, in the context of AMR, since the ASO have to reach the target mRNA to be efficient, the cellular uptake inside prokaryotic cells is a critical issue (Xue et al. (2018) Nanomedicine Nanotechnol Biol. Med. 14:745-758). Indeed, the carriers used in mammalian cells show much higher toxicity to bacterial cells and lower delivery efficacies.

There is thus an important need in identifying new solutions for efficiently targeting and fighting AMR.

The present invention meets this need.

The present invention arises from the unexpected finding by the inventors that antisense oligonucleotide sequences, in particular targeting the bla_{CTX-M15} gene, featuring a lipid moiety conjugated to the ASO extremity show a particularly efficient intracellular penetration in prokaryotic cells and that these lipid-modified antisense oligonucleotides can show a further improved enzymatic stability with phosphorothioate chemistry (PTO).

The present invention thus concerns an antisense oligonucleotide modified by substitution at the 5′ or the 3′ end by a lipid moiety, wherein said antisense oligonucleotide specifically targets an mRNA encoding a CTX-M extended-spectrum β-lactamase.

Another object of the invention concerns the antisense oligonucleotide of the invention for use for treating a bacterial infection, in particular due to bacteria resistant to 3^{rd} generation cephalosporins, 4^{th} generation cephalosporins and/or monobactams, in particular to 3^{rd} generation cephalosporins.

The present invention further concerns a pharmaceutical composition comprising (i) an antisense oligonucleotide of the invention, and (ii) a 3^{rd} generation cephalosporin, a 4^{th} generation cephalosporin and/or a monobactam.

Another object of the invention relative to the pharmaceutical composition of the invention for use for treating a bacterial infection, in particular due to bacteria resistant to 3^{rd} generation cephalosporins, 4^{th} generation cephalosporins and/or monobactams, in particular to 3^{rd} generation cephalosporins.

The present invention also concerns a kit comprising:
(i) an antisense oligonucleotide as defined in any one of claims 1 to 8, and
(ii) a 3^{rd} generation cephalosporin, a 4^{th} generation cephalosporin and/or a monobactam.

The present invention still concerns a kit of parts comprising:
(i) an antisense oligonucleotide as defined in any one of claims 1 to 8, and
(ii) a 3^{rd} generation cephalosporin, a 4^{th} generation cephalosporin and/or a monobactam.
   for separate, sequential and/or simultaneous administration to a subject.

Another object of the invention concerns the kit of the invention for use in a method for treating a bacterial infection in a subject, in particular a bacterial infection due to bacteria resistant to 3^{rd} generation cephalosporins, 4^{th} generation cephalosporins and/or monobactams, in particular to 3^{rd} generation cephalosporins, wherein (i) said antisense oligonucleotide and (ii) said 3^{rd} generation cephalosporin, 4^{th} generation cephalosporin and/or monobactam are administered separately, sequentially and/or simultaneously to the subject.

### DETAILED DESCRIPTION OF THE INVENTION

3^{rd} Generation Cephalosporins and CTX-M Extended Spectrum β-Lactamases

The present invention aims at fighting antimicrobial resistance, in particular antibiotic resistance.

By “antimicrobial resistance” or “AMR” is meant herein the phenomenon that a microorganism does not exhibit decreased viability or inhibited growth or reproduction when exposed to concentrations of the antimicrobial agent that can be attained with normal therapeutic dosage regimes in patients. It implies that an infection caused by this microorganism cannot be successfully treated with this antimicrobial agent.

As used herein, the terms “antibiotic” and “antimicrobial compound” are used interchangeably and refer to a compound which decreases the viability of a microorganism, or which inhibits the growth or reproduction of a microorganism.

In a particular embodiment, the antisense oligonucleotides, pharmaceutical compositions and kits of the invention aims at fighting bacterial resistance against 3^{rd} generation cephalosporin.

By “3^{rd} generation cephalosporin” is meant herein a β-lactam antibiotic, i.e. a compound with antibiotic properties containing a beta-lactam functionality, including but not limited to cefixime, ceftazidime, cefotaxime, ceftriaxone, cefcapene, cefdaloxime, cefdinir, cefditoren, cefetamet, cefmenoxime, cefodizime, cefoperazone, cefpimizole, cefpiramide, cefpodoxime, cefsulodin, cefteram, ceftibuten, ceftiolene, ceftizoxime, and oxacephem.

Preferably, said 3^{rd} generation cephalosporin is ceftriaxone.

By “4^{th} generation cephalosporin” is meant herein a β-lactam antibiotic, i.e. a compound with antibiotic properties containing a beta-lactam functionality, including but not limited to cefepime.

By “monobactam” is meant herein a subgroup of β-lactam antibiotics, which are monocyclic and wherein the β-lactam ring is not fused to another ring. They include aztreonam.

As well known from the skilled person, bacterial resistance against 3^{rd} generation cephalosporins is mainly due, in enterobacteria, to the presence of extended-spectrum β-lactamases (Bevan et al. (2017) J. Antimicrob. Chemother. 72:2145-2155 and Robin et al. (2017) Antimicrobial Agents and Chemotherapy 61:e01911-16). β-lactamases are a family of enzymes that hydrolyze β-lactam rings, such as β-lactam rings of β-lactam antibiotic drugs. β-lactamases are found in Gram positive and Gram negative bacteria and are responsible for the antibiotic resistance of many bacterial strains. β-lactamases can be classified on the basis of their primary structure into four molecular classes, namely classes A to D. Classes A, C and D have a serine residue at their active site and class B, or metallo-β-lactamases, have zinc at their active site. Carbapenemases are a diverse group of β-lactamases that include enzymes belonging to class A, B and D. Class A carbapenemases include KPC-1, KPC-2, KPC-3 and KPC-4. Class B carbapenemases include the IMP family, VIM family, GIM-1 and SPM-1 as well as others. Class D carbapenemases include OXA-23, OXA-24, OXA-25, OXA-26, OXA-27, OXA-40 and OXA-48 as well as others. AmpC β-lactamases are class C enzymes and can be encoded by chromosomal genes or be plasmid-borne. AmpC β-lactamases hydrolyze broad and extended-spectrum cephalosporins (i.e., cephamycins and oxyimino-beta lactams).

Extended-spectrum β-lactamases (ESBLs), which are targeted in the context of the present invention, are β-lactamases that hydrolyze cephalosporins with an oxyimino chain. ESBLs include the TEM family, SHV family as well as others, and the CTX-M family, which are class A enzymes.

In the context of the invention, the ESBLs specifically targeted by the antisense oligonucleotide of the invention are CTX-M ESBLs.

CTX-M ESBLs can be divided into five major groups, groups 1, 2, 8, 9 and 25, inside which sequence identities are high than 98%. Each group includes a number of minor allelic variants which differ from each other by one or few amino acid substitutions. Among these variants, the CTX-M-15 variant (belonging to group 1) is dominant worldwide.

Accordingly, in a particular embodiment, the CTX-M EBSL is a group 1 CTX-M ESBL.

Group 1 CTX-M ESBLs typically include CTX-M-1, CTX-M-3, CTX-M-10, CTX-M-11, CTX-M-12, CTX-M-15, CTX-M-22, CTX-M-23, CTX-M-28, CTX-M-29, CTX-M-30, CTX-M-32, CTX-M-33, CTX-M-34, CTX-M-36, CTX-M-37, CTX-M-42, CTX-M-52, CTX-M-53, CTX-M-54, CTX-M-55, CTX-M-57, CTX-M-58, CTX-M-60, CTX-M-61, CTX-M-62, CTX-M-66, CTX-M-68, CTX-M-69, CTX-M-71, CTX-M-72, CTX-M-79, CTX-M-80, CTX-M-88, CTX-M-96, CTX-M-101, CTX-M-103, CTX-M-107, CTX-M-108, CTX-M-109, CTX-M-114, CTX-M-116, CTX-M-117, CTX-M-133, CTX-M-136, CTX-M-139, CTX-M-142, CTX-M-144, CTX-M-150, CTX-M-155, CTX-M-156, CTX-M-157, CTX-M-158, CTX-M-162, CTX-M-163, CTX-M-164, CTX-M-169 and CTX-M-172 ESBLs.

In still a particular embodiment, said CTX-M ESBL is the CTX-M-15 ESBL.

The CTX-M-15 ESBL is encoded by the bla_{CTX-M15} gene. The Escherichia coli CTX-M-15 coding sequence consists typically of the sequence SEQ ID NO: 5. The Escherichia coli CTX-M-15 amino acid sequence consists typically of the sequence SEQ ID NO: 6.

In Escherichia coli plasmid, the bla_{CTX-M-15} gene is typically preceded by an associated upstream insertional element ISEcp1. The nucleic acid sequence of the Escherichia coli bla_{CTX-M-15} gene preceded by the associated upstream insertional element ISEcp1 is typically of sequence SEQ ID NO: 7.

### Antisense Oligonucleotide

As used herein, the term “oligonucleotide” refers to a nucleic acid sequence which may be 3′-5′ or 5′-3′ oriented. The oligonucleotide of the invention may in particular be DNA or RNA. In a particular embodiment, the oligonucleotide used in the context of the invention is DNA.

The oligonucleotide of the invention preferably comprises or consists of a nucleic acid sequence, in particular a DNA sequence, of at least 15 nucleotides, preferably at least 18 nucleotides, at least 19 nucleotides, at least 20 nucleotides, at least 21 nucleotides, at least 22 nucleotides, at least 23 nucleotides, at least 24 nucleotides or at least 25 nucleotides. In a preferred embodiment, the oligonucleotide of the invention comprises or consists of a nucleic acid sequence, in particular a DNA sequence, of at least 19 nucleotides. In another preferred embodiment, the oligonucleotide of the invention comprises or consists of a nucleic acid sequence, in particular a DNA sequence, of less than 25 nucleotides. In a particularly preferred embodiment of the invention, the oligonucleotide of the invention comprises or consists of a nucleic acid sequence, in particular a DNA sequence, of at least 19 nucleotides and less than 25 nucleotides.

In a particular embodiment, the oligonucleotide of the invention comprises or consists of a nucleic acid sequence, in particular a DNA sequence, of 19 nucleotides, 20 nucleotides, 21 nucleotides or 25 nucleotides.

The oligonucleotides of the invention may be further modified (in addition to the lipid modification), preferably chemically modified, in order to increase the stability of the oligonucleotides in vivo. In particular, the oligonucleotide of the invention may comprise modified nucleotides.

Chemical modifications may occur at three different sites: (i) at phosphate groups, (ii) on the sugar moiety, and/or (iii) on the entire backbone structure of the oligonucleotide.

For example, the oligonucleotides may be employed as phosphorothioate derivatives (replacement of a non-bridging phosphoryl oxygen atom with a sulfur atom) which have increased resistance to nuclease digestion. 2′-methoxyethyl (MOE) modification (such as the modified backbone commercialized by ISIS Pharmaceuticals) is also effective.

In a particular embodiment, the antisense oligonucleotide of the invention is a phosphorothioate derivative.

Additionally or alternatively, the oligonucleotides of the invention may comprise completely, partially or in combination, modified nucleotides which are derivatives with substitutions at the 2′ position of the sugar, in particular with the following chemical modifications: O-methyl group (2′-O-Me) substitution, 2-methoxyethyl group (2′-O-MOE) substitution, fluoro group (2′-fluoro) substitution, chloro group (2′-Cl) substitution, bromo group (2′-Br) substitution, cyanide group (2′-CN) substitution, trifluoromethyl group (2′-CF₃) substitution, OCF₃ group (2′-OCF₃) substitution, OCN group (2′-OCN) substitution, O-alkyl group (2′-O-alkyl) substitution, S-alkyl group (2′-S-alkyl) substitution, N-alkyl group (2′-N-alkyl) substitution, O-alkenyl group (2′-O-alkenyl) substitution, S-alkenyl group (2′-S-alkenyl) substitution, N-alkenyl group (2′-N-alkenyl) substitution, SOCH₃ group (2′-SOCH₃) substitution, SO₂CH₃ group (2′-SO₂CH₃) substitution, ONO₂ group (2′-ONO₂) substitution, NO₂ group (2′-NO₂) substitution, N₃ group (2′-N₃) substitution and/or NH₂ group (2′-NH₂) substitution.

Additionally or alternatively, the oligonucleotides of the invention may comprise completely or partially modified nucleotides wherein the ribose moiety is used to produce locked nucleic acid (LNA), in which a covalent bridge is formed between the 2′ oxygen and the 4′ carbon of the ribose, fixing it in the 3′-endo configuration. These constructs are extremely stable in biological medium, able to activate RNase H and form tight hybrids with complementary RNA and DNA.

Accordingly, in a preferred embodiment, the oligonucleotide of the invention comprises modified nucleotides selected from the group consisting of LNA, 2′-OMe analogs, 2′-phosphorothioate analogs, 2′-fluoro analogs, 2′-Cl analogs, 2′-Br analogs, 2′-CN analogs, 2′-CF₃ analogs, 2′-OCF₃ analogs, 2′-OCN analogs, 2′-O-alkyl analogs, 2′-S-alkyl analogs, 2′-N-alkyl analogs, 2′-O-alkenyl analogs, 2′-S-alkenyl analogs, 2′-N-alkenyl analogs, 2′-SOCH₃ analogs, 2′-SO₂CH₃ analogs, 2′-ONO₂ analogs, 2′-NO₂ analogs, 2′-N₃ analogs, 2′-NH₂ analogs and combinations thereof. More preferably, the modified nucleotides are selected from the group consisting of LNA, 2′-OMe analogs, 2′-phosphorothioate analogs and 2′-fluoro analogs.

In a particular embodiment, the oligonucleotide of the invention is a LNA-PTO gapmer.

Additionally or alternatively, some nucleobases of the oligonucleotide may be present as desoxyriboses. That modification should only affect the skeleton of the nucleobase, in which the hydroxyl group is absent, but not the side chain of the nucleobase which remains unchanged.

The oligonucleotide of the invention are antisense oligonucleotides which target mRNAs encoding a CTX-M extended spectrum β-lactamase as defined above.

As used herein, the term “antisense oligonucleotide” refers to a single stranded DNA or RNA with complementary sequence to its target mRNA, and which binds its target mRNA thereby preventing protein translation either by steric hindrance of the ribosomal machinery or induction of mRNA degradation by ribonuclease H.

The antisense oligonucleotide may be a DNA or a RNA molecule.

As used herein, an oligonucleotide that “targets” an mRNA refers to an oligonucleotide that is capable of specifically binding to said mRNA. That is to say, the oligonucleotide comprises a sequence that is at least partially complementary, preferably perfectly complementary, to a region of the sequence of said mRNA, said complementarity being sufficient to yield specific binding under intra-cellular conditions.

As immediately apparent to the skilled in the art, by a sequence that is “perfectly complementary to” a second sequence is meant the reverse complement counterpart of the second sequence, either under the form of a DNA molecule or under the form of a RNA molecule. A sequence is “partially complementary to” a second sequence if there are one or more mismatches.

Preferably, the antisense oligonucleotide of the invention is capable of reducing the amount of CTX-M extended spectrum β-lactamase in bacteria.

Nucleic acids that target an mRNA encoding a CTX-M extended spectrum β-lactamase may be designed by using the sequence of said mRNA as a basis, e.g. using bioinformatic tools. For example, the sequences of SEQ ID NO: 5, SEQ ID NO: 7 or SEQ ID NO: 8 can be used as a basis for designing nucleic acids that target an mRNA encoding a CTX-M extended spectrum β-lactamase.

Preferably, the antisense oligonucleotides of the invention are capable of reducing the amount of CTX-M extended spectrum β-lactamase in bacteria, e.g. the amount of CTX-M-15 extended spectrum β-lactamase in bacterial cells such as Escherichia coli TcK12 cells.

Methods for determining whether an oligonucleotide is capable of reducing the amount of CTX-M extended spectrum β-lactamase in cells are known to the skilled in the art. This may be done for example by analyzing β-lactamase activity by hydrolyzing nitrocefin, a chromogenic cephalosporin, in the presence and in the absence of the oligonucleotide to be tested (see Examples).

In particular, the inventors have designed four antisense oligonucleotides targeting an mRNA encoding CTX-M extended-spectrum β-lactamase that are very efficient in reducing the amount of CTX-M extended spectrum β-lactamase in bacteria. These oligonucleotides target the region situated between nucleotide −4 upstream the atg codon and nucleotide 21 of the CTX-M coding sequence, the region situated between nucleotides 498 and 504 of the CTX-M coding sequence, the region situated between nucleotides 4 and 28 of the CTX-M coding sequence, and the region situated between nucleotides 492 and 512 of the CTX-M coding sequence, respectively.

The inventors have designed 3 additional antisense oligonucleotides targeting an mRNA encoding CTX-M extended-spectrum β-lactamase that decrease the ceftriaxone Minimal Inhibitory Concentration (MIC) in resistant laboratory E. coli strain TcK12. These oligonucleotides target the region situated between nucleotides 53 and 75 of the CTX-M coding sequence, the region situated between nucleotides 480 and 500 of the CTX-M coding sequence and the region situated between nucleotides 781 and 805 of the CTX-M coding sequence, respectively.

Therefore, the oligonucleotides according to the invention preferably target a sequence overlapping with nucleotides 38 to 62 of SEQ ID NO: 8, or with nucleotides 498 to 504 of SEQ ID NO: 5, or with nucleotides 4 to 28 of SEQ ID NO: 5, or with nucleotides 492 to 512 of SEQ ID NO: 5, or with nucleotides 53 to 75 of SEQ ID NO: 5, or with nucleotides 480 to 500 of SEQ ID NO: 5 or with nucleotides 781 to 805 of SEQ ID NO: 1, said oligonucleotide being a DNA or a RNA.

More preferably, the oligonucleotides according to the invention target a sequence overlapping with nucleotides 38 to 62 of SEQ ID NO: 8, or with nucleotides 498 to 504 of SEQ ID NO: 5, or with nucleotides 4 to 28 of SEQ ID NO: 5, or with nucleotides 492 to 512 of SEQ ID NO: 5, said oligonucleotide being a DNA or a RNA.

The oligonucleotides of the invention may for example consist of a sequence selected from the group consisting of the sequences GCGCAGTGATTTTTTAACCATGGGA (SEQ ID NO: 1), CGTGTAGGTACGGCAGATC (SEQ ID NO: 2), TGAACTGGCGCAGTGATTTTTTAAC (SEQ ID NO: 3), GTCGGCTCGGTACGGTCGAGA (SEQ ID NO: 4), CGGCACACTTCCTAACAACA (SEQ ID NO: 10), ACGGTCGAGACGGAACGTTT (SEQ ID NO: 11) and AGGCTGGGTGAAGTAAGTGA (SEQ ID NO: 12).

Preferably, the oligonucleotides of the invention consist of a sequence selected from the group consisting of the sequences GCGCAGTGATTTTTTAACCATGGGA (SEQ ID NO: 1), CGTGTAGGTACGGCAGATC (SEQ ID NO: 2), TGAACTGGCGCAGTGATTTTTTAAC (SEQ ID NO: 3) and GTCGGCTCGGTACGGTCGAGA (SEQ ID NO: 4).

### Lipid Modification

The antisense oligonucleotide of the invention is an antisense oligonucleotide as defined above, modified by substitution at the 5′ or the 3′ end by a lipid moiety.

In the context of the invention, the term “lipid moiety” refers to a moiety having at least one lipid. Lipids are small molecules having hydrophobic or amphiphilic properties and are useful for preparation of vesicles, micelles and liposomes. Lipids include, but are not limited to, fats, waxes, fatty acids, cholesterol, phospholipids, monoglycerides, diglycerides, triglycerides and highly fluorinated chains.

In a preferred embodiment of the invention, the lipid moiety is a moiety comprising at least one ketal functional group, wherein the ketal carbon of said ketal functional group bears two saturated or unsaturated, linear or branched, hydrocarbon chains comprising from 1 to 22 carbon atoms, preferably from 6 to 20 carbon atoms, more preferably from 10 to 18 carbon atoms or from 12 to 15 carbon atoms.

In a particular embodiment of the invention, the modified antisense oligonucleotide of the invention is of the general formula (I)

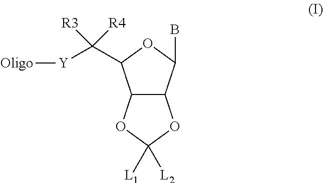

wherein:
Oligo represents an antisense oligonucleotide sequence as defined in the section “Antisense oligonucleotide” above, wherein said antisense oligonucleotide may be oriented 3′-5′ or 5′-3′, simple stranded, DNA, RNA, and/or comprise modified nucleotides;
Y represents a divalent linker moiety selected from ether —O—, thio —S—, amino —NH—, and methylene —CH₂—;
R₃ and R₄ may be identical or different and represent:
   a hydrogen atom,
   a halogen atom, in particular fluorine atom,
   a hydroxyl group, or
   an alkyl group comprising from 1 to 12 carbon atoms;
L₁ and L₂ may be identical or different and represent a saturated or unsaturated, linear or branched hydrocarbon chain comprising from 1 to 22 carbon atoms;
B is an optionally substituted nucleobase, selected from the group consisting of purine nucleobases, pyrimidine nucleobases, and non-natural monocyclic or bicyclic heterocyclic nucleobases wherein each cycle comprises from 4 to 7 atoms.

In the context of the invention, the term “alkyl” refers to a hydrocarbon chain that may be a linear or branched chain, containing the indicated number of carbon atoms. For example, C₁-C₁₂ alkyl indicates that the group may have from 1 to 12 (inclusive) carbon atoms in it.

Preferably, the antisense oligonucleotide sequence “Oligo-” is connected to the divalent linker moiety Y via a phosphate-O—P(═O)(O⁻)— or a phosphorothioate —O—P(═S)(0-)— moiety, at its 3′ or 5′ end, advantageously at its 5′ end.

In a preferred embodiment of the invention, the modified antisense oligonucleotide is of the general formula (I′):

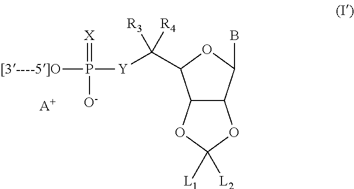

wherein:
wherein Y, R₃, R₄, L₁, L₂ and B are as defined above in formula (I),
X represents O or S,
[3′----5′] represents, along with the —O—P(═O)(O⁻)— or the —O—P(═S)(O⁻)— residue, an antisense oligonucleotide as defined in the section “Antisense oligonucleotide” herein above, and
A⁺ represents a cation, preferably H⁺, Na⁺, K⁺ or NH₄⁺.

In the formulae (I) and (I′), the divalent linker moiety Y is preferably ether —O—.

In the formulae (I) and (I′), R₃ and R₄ are preferably hydrogen atoms.

In a preferred embodiment of the invention, the modified antisense oligonucleotide is of the formula (I″):

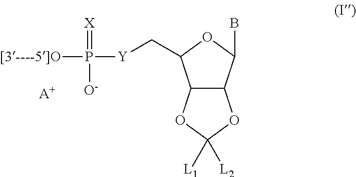

wherein Y, L₁, L₂ and B are as defined above in formula (I), X and A⁺ are as defined above in formula (I′) and [3′----5′] represents, along with the O—P(═O)(O⁻)— or the —O—P(═S)(O⁻)— residue, an antisense oligonucleotide as defined in the section “Antisense oligonucleotide” herein above.

In the formulae (I), (I′) and (I″), L₁ and L₂ preferably represent a hydrocarbon chain, preferably a linear hydrocarbon chain, comprising from 6 to 22 carbon atoms, preferably from 8 to 18 carbon atoms, advantageously from 12 to 16 carbon atoms, more advantageously 15 carbon atoms.

In the formulae (I), (I′) and (I″), B preferably represents a non substituted nucleobase selected from the group consisting of uracil, thymine, adenine, guanine, cytosine, 6-methoxypurine, 7-methylguanine, xanthine, 5,6-dihydrouracil, 5-methylcytosine, 5-hydroxymethylcytosine and hypoxanthine.

Preferably, in the formulae (I), (I′) and (I″), B represents a non substituted nucleobase selected from the group consisting of uracil, thymine, adenine, cytosine, 6-methoxypurine and hypoxanthine.

More preferably, in the formulae (I), (I′) and (I″), B represents uracil.

In the formulae (I′) and (I″), X preferably represents S.

In a preferred embodiment of the invention, the modified antisense oligonucleotide is of the formula (I′″):

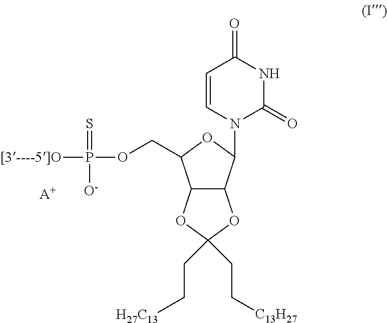

wherein A⁺ is as defined above in formula (I′) and [3′----5′] represents, along with the —O—P(═S)(O⁻)— residue, an antisense oligonucleotide as defined in the section “Antisense oligonucleotide” herein above.

In another particular embodiment, the lipid moiety is a moiety comprising at least one saturated or unsaturated, linear or branched hydrocarbon chain comprising from 2 to 60 carbon atoms, preferably from 2 to 40 carbon atoms, still preferably from 2 to 30 carbon atoms, preferably from 5 to 20 carbon atoms, more preferably from 10 to 18 carbon atoms.

In a particular embodiment, the modified antisense oligonucleotide is of the general formula (II)

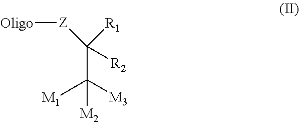

wherein:
Oligo is a defined above concerning formula (I);
Z represents a divalent linker moiety selected from ether —O—, thio —S—, amino —NH—, and methylene —CH₂—;
R₁ and R₂ may be identical or different and represent:
   a hydrogen atom,
   a halogen atom, in particular fluorine atom,
   a hydroxyl group, or
   an alkyl group comprising from 1 to 12 carbon atoms;
M₁, M₂ and M₃ may be identical or different and represent:
   a hydrogen atom,
   a saturated or unsaturated, linear or branched hydrocarbon chain comprising from 2 to 30 carbon atoms, preferably from 6 to 22 carbon atoms, more preferably from 12 to 20 carbon atoms, which may be substituted by one or more halogen atoms, notably be fluorinated or prefluorinated and/or be interrupted by one or more groups selected from ether —O—, thio —S—, amino —NH—, oxycarbonyl —O—C(O)—, thiocarbamate —O—C(S)—NH—, carbonate —O—C(O)—O—, carbamate —O—C(O)—NH—, phosphate —O—P(O)(O)—O— and phosphonate —P—O(O)(O)— groups; and/or be substituted at the terminal carbon atom by an aliphatic or aromatic, notably benzylic or naphtylic ester or ether group;
   an acyl radical with 2 to 30 carbon atoms, or
   an acylglycerol, sphingosine or ceramide group, provided that at least one of M₁, M₂ and M₃ is not a hydrogen atom.

In the context of the invention, the term “acyl” refers to an alkylcarbonyl, cycloalkylcarbonyl, arylcarbonyl, heterocyclylcarbonyl or heteroarylcarbonyl substituent.

Preferably, the oligonucleotide sequence “Oligo-” is connected to the divalent linker moiety Z via a phosphate moiety —O—P(═O)(O⁻)— or a phosphorothioate —O—P(═S)(0-)— moiety, at its 3′ or 5′ end, advantageously at its 5′ end.

In a particular embodiment according to the invention, the modified antisense oligonucleotide is of the general formula (II′):

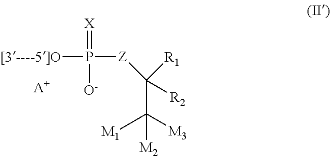

wherein:
Z, R₁, R₂, M₁, M₂ and M₃ are as defined above in formula (II),
X is as defined above in formula (I′) and (I″),
[3′----5′] is as defined above in formula (I′) and (I″), and -A⁺ represents a cation, preferably H⁺, Na⁺, K⁺ or NH₄⁺.

In the formulae (II) and (II′), the divalent linker moiety Z is preferably ether —O—.

In the formulae (II) and (II′), R₁ and R₂ are preferably hydrogen atoms.

In a particular embodiment according to the invention, the modified antisense oligonucleotide is of the formula (II″):

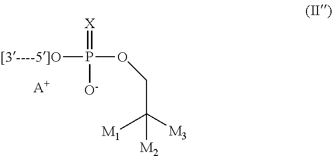

wherein M₁, M₂ and M₃ are as defined above in formula (II), A⁺ is as defined above in formula (II′) and [3′----5′] is as defined above in formula (I′) and (I″).

In the formulae (II), (II′) and (II″), M₁, M₂ and M₃ preferably represent a hydrocarbon chain, preferably a linear hydrocarbon chain, comprising from 6 to 22 carbon atoms, preferably from 12 to 20 carbon atoms, more preferably 18 carbon atoms.

In the formulae (II′) and (II″), X preferably represents O.

In a particular embodiment according to the invention, the modified antisense oligonucleotide is of the formula (II′″):

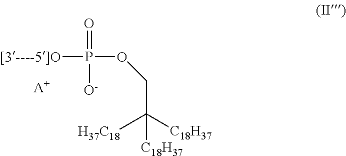

wherein A⁺ is as defined above in formula (II′) and [3′----5′] represents, along with the-O—P(═O)(O⁻)— residue, an antisense oligonucleotide as defined in the section “Antisense oligonucleotide” herein above.

### Pharmaceutical Composition

The present invention also concerns a pharmaceutical composition comprising (i) an antisense oligonucleotide of the invention, and (ii) a 3^{rd} generation cephalosporin, a 4^{th} generation cephalosporin and/or a monobactam, as defined in the section “3^{rd} generation cephalosporins and CTX-M extended spectrum β-lactamases” above, in particular a 3^{rd} generation cephalosporin as defined in the section “3^{rd} generation cephalosporins and CTX-M extended spectrum β-lactamases” above.

The pharmaceutical composition of the invention may further comprise a pharmaceutically acceptable excipient.

The term “pharmaceutically acceptable” refers to properties and/or substances which are acceptable for administration to a subject from a pharmacological or toxicological point of view. Further “pharmaceutically acceptable” refers to factors such as formulation, stability, patient acceptance and bioavailability which will be known to a manufacturing pharmaceutical chemist from a physical/chemical point of view.

As used herein, “pharmaceutically acceptable excipient” refers to any substance in a pharmaceutical composition different from the active ingredient. Said excipients can be liquids, sterile, as for example water and oils, including those of origin in the petrol, animal, vegetable or synthetic, as peanut oil, soy oil, mineral oil, sesame oil, and similar, disintegrate, wetting agents, solubilizing agents, antioxidant, antimicrobial agents, isotonic agents, stabilizing agents or diluents. Suitable adjuvants and/or pharmaceutical carriers are described in “Remington's Pharmaceutical Sciences” by E. W. Martin.

The pharmaceutical compositions of the invention can be formulated for a parenteral (e.g., intravascular, intradermal, intracerebroventricular, subcutaneous, intramuscular, intraperitoneal), oral, buccal, nasal and pulmonary, other transmucosal (eg., vaginal, rectal), transdermal, topical, or intraocular administration, for local or systemic effect.

### Kits

Still another object of the invention is a kit comprising:
(i) an antisense oligonucleotide of the invention, and
(ii) a 3^{rd} generation cephalosporin, a 4^{th} generation cephalosporin and/or a monobactam, as defined in the section “3^{rd} generation cephalosporins and CTX-M extended spectrum β-lactamases” above, in particular a 3^{rd} generation cephalosporin as defined in the section “3^{rd} generation cephalosporins and CTX-M extended spectrum β-lactamases” above.

Said (i) antisense oligonucleotide and said (ii) 3^{rd} generation cephalosporin, 4^{th} generation cephalosporin and/or monobactam, may respectively be formulated in a pharmaceutical composition, each pharmaceutical composition respectively optionally further comprising a pharmaceutically acceptable excipient as defined in the section “Pharmaceutical composition” above.

The present invention still concerns a kit of parts comprising:
(i) an antisense oligonucleotide of the invention, and
(ii) a 3^{rd} generation cephalosporin, a 4^{th} generation cephalosporin and/or a monobactam, as defined in the section “3^{rd} generation cephalosporins and CTX-M extended spectrum β-lactamases” above, in particular a 3^{rd} generation cephalosporin as defined in the section “3^{rd} generation cephalosporins and CTX-M extended spectrum β-lactamases” above,
   for separate, sequential and/or simultaneous administration to a subject, as defined in the section “Medical indications” below.

The (i) antisense oligonucleotides of the invention and the (ii) 3^{rd} generation cephalosporin 4^{th} generation cephalosporin and/or monobactam can be respectively and independently administered by any suitable route, in particular by parenteral (e.g., intravascular, intradermal, intracerebroventricular, subcutaneous, intramuscular, intraperitoneal), oral, buccal, nasal and pulmonary, other transmucosal (eg., vaginal, rectal), transdermal, topical, or intraocular route, for local or systemic effect.

### Medical Indications

The present invention concerns the antisense oligonucleotide of the invention, for use for treating a bacterial infection.

In a particular embodiment, said antisense oligonucleotide is for use in combination with a 3^{rd} generation cephalosporin, 4^{th} generation cephalosporin and/or monobactam as defined in the section “3^{rd} generation cephalosporins and CTX-M extended spectrum β-lactamases” above.

Another object of the invention concerns the use of an antisense oligonucleotide of the invention for the manufacture of a medicament intended for treating a bacterial infection.

In a particular embodiment, said medicament is to be used in combination with a 3^{rd} generation cephalosporin, 4^{th} generation cephalosporin and/or monobactam as defined in the section “3^{rd} generation cephalosporins and CTX-M extended spectrum β-lactamases” above.

Still another object of the invention concerns a method of treating a bacterial infection in a subject, said method comprising the administration of a therapeutically effective amount of an antisense oligonucleotide of the invention in a subject in need thereof.

In a particular embodiment, said method comprises the combined administration, in said subject, of a 3^{rd} generation cephalosporin, 4^{th} generation cephalosporin and/or monobactam as defined in the section “3^{rd} generation cephalosporins and CTX-M extended spectrum β-lactamases” above.

The present invention also concerns the pharmaceutical composition of the invention, for use for treating a bacterial infection.

Another object of the invention concerns the use of (i) an antisense oligonucleotide of the invention and of (ii) a 3^{rd} generation cephalosporin, 4^{th} generation cephalosporin and/or monobactam, as defined in the section “3^{rd} generation cephalosporins and CTX-M extended spectrum β-lactamases” above for the manufacture of a pharmaceutical composition intended for treating a bacterial infection.

Still another object of the invention concerns a method of treating a bacterial infection in a subject, said method comprising the administration of a therapeutically effective amount of a pharmaceutical composition of the invention in a subject in need thereof.

The present invention also concerns a kit of the invention for use in a method for treating a bacterial infection in a subject, wherein said (i) antisense oligonucleotide and said (ii) 3^{rd} generation cephalosporin, 4^{th} generation cephalosporin and/or monobactam are administered separately, sequentially and/or simultaneously to the subject.

Another object of the invention concerns the use of (i) an antisense oligonucleotide of the invention and of (ii) a 3^{rd} generation cephalosporin, 4^{th} generation cephalosporin and/or monobactam, as defined in the section “3^{rd} generation cephalosporins and CTX-M extended spectrum β-lactamases” above for the manufacture of a combined pharmaceutical preparation intended for treating a bacterial infection in a subject, wherein said (i) antisense oligonucleotide and said (ii) 3^{rd} generation cephalosporin, 4^{th} generation cephalosporin and/or monobactam, are administered separately, sequentially and/or simultaneously to the subject.

Still another object of the invention concerns a method of treating a bacterial infection in a subject, said method comprising the separate, sequential and/or simultaneous administration of a therapeutically effective amount of (i) an antisense oligonucleotide of the invention and of (ii) a 3^{rd} generation cephalosporin, 4^{th} generation cephalosporin and/or monobactam, as defined in the section “3^{rd} generation cephalosporins and CTX-M extended spectrum β-lactamases” in a subject in need thereof.

In particular embodiments of the invention the bacterial infection to be treated is due to bacteria resistant to 3^{rd} generation cephalosporins, 4^{th} generation cephalosporins and/or monobactams.

By “bacteria resistant to 3^{rd} generation cephalosporins, 4^{th} generation cephalosporins and/or monobactams” is meant bacteria producing ESBLs, as defined in the section “3^{rd} generation cephalosporins and CTX-M extended spectrum β-lactamases” above.

Accordingly, in particular embodiments, said bacteria carry a bla_{CTX-M} gene as defined in the section “3^{rd} generation cephalosporins and CTX-M extended spectrum β-lactamases” above, in particular a Group 1 bla_{CTX-M} gene, more particularly a bla_{CTX-M-15} gene.

In still particular embodiments, said bacteria are Gram negative bacteria in particular resistant to 3^{rd} generation cephalosporins, 4^{th} generation cephalosporins and/or monobactams, in particular carrying a bla_{CTX-M} gene as defined above.

By way of Gram-negative bacteria, mention may be made of bacteria of the members of the order ‘Enterobacteriales’ and of the new reported order Enterobacterales ord. nov. which comprises seven families: Enterobacteriaceae, Erwiniaceae fam. nov., Pectobacteriaceae fam. nov., Yersiniaceae fam. nov., Hafniaceae fam. nov., Morganellaceae fam. nov., and Budviciaceae fam. nov.

In particular embodiments, said Gram-negative bacteria are selected from Escherichia, Salmonella, Shigella, Klebsiella, Serratia, Proteus, Morganella, Yersinia, Citrobacter, Hafnia, Edwardsiella, Providencia, Cedecea, Erwinia and Pantoea,

In still particular embodiments, said bacterial infection to be treated is due to Enterobacteriaceae bacteria, in particular resistant to 3^{rd} generation cephalosporins, 4^{th} generation cephalosporins and/or monobactams, in particular carrying a bla_{CTX-M} gene as defined above.

Enterobacteriaceae bacteria include bacteria of the genera Escherichia, Ewingella, Hafnia, Klebsiella, Kluyvera, Leclercia, Rahnella, Salmonella, and Shigella.

In more particular embodiments, said bacterial infection to be treated is due to bacteria of the genera Escherichia or Klebsiella, in particular resistant to 3^{rd} generation cephalosporins, 4^{th} generation cephalosporins and/or monobactams, in particular carrying a bla_{CTX-M} as defined above.

In still particular embodiments, said bacterial infection to be treated is due to bacteria of the Escherichia coli or the Klebsiella pneumoniae species, in particular resistant to 3^{rd} generation cephalosporins, 4^{th} generation cephalosporins and/or monobactams, in particular carrying a bla_{CTX-M} as defined above.

In particularly preferred embodiments, said bacterial infection to be treated is due to bacteria of the Escherichia coli species, in particular resistant to 3^{rd} generation cephalosporins, 4^{th} generation cephalosporins and/or monobactams, in particular carrying a bla_{CTX-M} as defined above.

By “subject” is meant herein a mammal, such as a rodent, a feline, a canine, or a primate. Preferably, a subject according to the invention is a human.

In the context of the invention, the term “treating” or “treatment” means reversing, alleviating, inhibiting the progress of the disorder or condition to which such term applies, or one or more symptoms of such disorder or condition.

By a “therapeutically effective amount” of an antisense oligonucleotide or a pharmaceutical composition of the invention or a 3^{rd} generation cephalosporin is meant a sufficient amount of the antisense oligonucleotide or composition or cephalosporin to treat a specific disease, at a reasonable benefit/risk ratio applicable to any medical treatment. It will be understood, however, that the total daily usage of the antisense oligonucleotide or composition of the present invention or cephalosporin will be decided by the attending physician within the scope of sound medical judgment. The specific therapeutically effective dose level for any particular subject will depend upon a variety of factors including the disorder being treated and the severity of the disorder, activity of the specific antisense oligonucleotides or compositions or cephalosporins employed, the specific combinations employed, the age, body weight, general health, sex and diet of the subject, the time of administration, route of administration and rate of excretion of the specific compounds employed, the duration of the treatment, drugs used in combination or coincidental with the specific compounds employed, and like factors well known in the medical arts. For example, it is well within the skill of the art to start doses of the antisense oligonucleotides at levels lower than those required to achieve the desired therapeutic effect and to gradually increase the dosage until the desired effect is achieved.

The form of the pharmaceutical compositions, the route of administration, the dosage and the regimen naturally depend upon the condition to be treated, the severity of the illness, the age, weight, and sex of the patient, etc.

The antisense oligonucleotides and pharmaceutical compositions of the invention and/or 3^{rd} generation cephalosporins, 4^{th} generation cephalosporins and/or monobactams can be administered by any suitable route, in particular by parenteral (e.g., intravascular, intradermal, intracerebroventricular, subcutaneous, intramuscular, intraperitoneal), oral, buccal, nasal and pulmonary, other transmucosal (eg., vaginal, rectal), transdermal, topical, or intraocular route, for local or systemic effect.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

The present invention will be further illustrated by the figures and examples below.

| SEQ | | |
|---|---|---|
| ID | Description | Sequence |
| 1 | Nucleotide | GCGCAGTGATTTTTTAACCA |
| | sequence of | TGGGA |
| | the LASO_{α} | |
| | antisense | |
| | oligonucleotide | |
| 2 | Nucleotide | CGTGTAGGTACGGCAGATC |
| | sequence of | |
| | the LASO_{β} | |
| | antisense | |
| | oligonucleotide | |
| 3 | Nucleotide | TGAACTGGCGCAGTGATTTT |
| | sequence of | TTAAC |
| | the LASO_{γ} | |
| | antisense | |
| | oligonucleotide | |
| 4 | Nucleotide | GTCGGCTCGGTACGGTCGAG |
| | sequence of | A |
| | the LASO_{δ} | |
| | antisense | |
| | oligonucleotide | |
| 5 | Escherichia coli | atggttaaaaaatcactgcg |
| | CTX-M-15 coding | ccagttcacgctgatggcga |
| | sequence | cggcaaccgtcacgctgttg |
| | | ttaggaagtgtgccgctgta |
| | | tgcgcaaacggcggacgtac |
| | | agcaaaaacttgccgaatta |
| | | gagcggcagtcgggaggcag |
| | | actgggtgtggcattgatta |
| | | acacagcagataattcgcaa |
| | | atactttatcgtgctgatga |
| | | gcgctttgcgatgtgcagca |
| | | ccagtaaagtgatggccgcg |
| | | gccgcggtgctgaagaaaag |
| | | tgaaagcgaaccgaatctgt |
| | | taaatcagcgagttgagatc |
| | | aaaaaatctgaccttgttaa |
| | | ctataatccgattgcggaaa |
| | | agcacgtcaatgggacgatg |
| | | tcactggctgagcttagcgc |
| | | ggccgcgctacagtacagcg |
| | | ataacgtggcgatgaataag |
| | | ctgattgctcacgttggcgg |
| | | cccggctagcgtcaccgcgt |
| | | tcgcccgacagctgggagac |
| | | gaaacgttccgtctcgaccg |
| | | taccgagccgacgttaaaca |
| | | ccgccattccgggcgatccg |
| | | cgtgataccacttcacctcg |
| | | ggcaatggcgcaaactctgc |
| | | ggaatctgacgctgggtaaa |
| | | gcattgggcgacagccaacg |
| | | ggcgcagctggtgacatgga |
| | | tgaaaggcaataccaccggt |
| | | gcagcgagcattcaggctgg |
| | | actgcctgcttcctgggttg |
| | | tgggggataaaaccggcagc |
| | | ggtggctatggcaccaccaa |
| | | cgatatcgcggtgatctggc |
| | | caaaagatcgtgcgccgctg |
| | | attctggtcacttacttcac |
| | | ccagcctcaacctaaggcag |
| | | aaagccgtcgcgatgtatta |
| | | gcgtcggcggctaaaatcgt |
| | | caccgacggtttgtaa |
| 6 | Escherichia coli | MVKKSLRQFTLMATATVTLL |
| | CTX-M-15 | LGSVPLYAQTADVQQKLAEL |
| | amino acid | ERQSGGRLGVALINTADNSQ |
| | sequence | ILYRADERFAMCSTSKVMAA |
| | | AAVLKKSESEPNLLNQRVEI |
| | | KKSDLVNYNPIAEKHVNGTM |
| | | SLAELSAAALQYSDNVAMNK |
| | | LIAHVGGPASVTAFARQLGD |
| | | ETFRLDRTEPTLNTAIPGDP |
| | | RDTTSPRAMAQTLRNLTLGK |
| | | ALGDSQRAQLVTWMKGNTTG |
| | | AASIQAGLPASWVVGDKTGS |
| | | GGYGTTNDIAVIWPKDRAPL |
| | | ILVTYFTQPQPKAESRRDVL |
| | | ASAAKIVTDGL |
| 7 | nucleic acid | tgggatttttgattttattg |
| | sequence of | aaaatgacctcgtatttgat |
| | the Escherichia coli | aatgactcaacaaataaaat |
| | bla_{CTX-M-15} | caagatgaatcatataaaga |
| | gene preceded | ccatgctctgcggtcacttc |
| | by the associated | attggcattgataagttaga |
| | upstream | acgtctaaagctacttcaaa |
| | insertional | atgatcccctcgtcaacgag |
| | element ISEcp1 | tttgatatttccgtaaaaga |
| | | acctgaaacagtgtcacggt |
| | | ttctaggaaacttcaacttc |
| | | aagacaacccaaatgtttag |
| | | agacattaattttaaagtct |
| | | ttaaaaaactgctcactaaa |
| | | agtaaattgacatccattac |
| | | gattgatattgatagtagtg |
| | | taattaacgtagaaggtcat |
| | | caagaaggtgcgtcaaaagg |
| | | atataatcctaagaaactgg |
| | | gaaaccgatgctacaatatc |
| | | caatttgcattttgcgacga |
| | | attaaaagcatatgttaccg |
| | | gatttgtaagaagtggcaat |
| | | acttacactgcaaacggtgc |
| | | tgcggaaatgatcaaagaaa |
| | | ttgttgctaacatcaaatca |
| | | gacgatttagaaattttatt |
| | | tcgaatggatagtggctact |
| | | ttgatgaaaaaattatcgaa |
| | | acgatagaatctcttggatg |
| | | caaatatttaattaaagcca |
| | | aaagttattctacactcacc |
| | | tcacaagcaacgaattcatc |
| | | aattgtattcgttaaaggag |
| | | aagaaggtagagaaactaca |
| | | gaactgtatacaaaattagt |
| | | taaatgggaaaaagacagaa |
| | | gatttgtcgtatctcgcgta |
| | | ctgaaaccagaaaaagaaag |
| | | agcacaattatcacttttag |
| | | aaggttccgaatacgactac |
| | | tttttctttgtaacaaatac |
| | | taccttgctttctgaaaaag |
| | | tagttatatactatgaaaag |
| | | cgtggtaatgctgaaaacta |
| | | tatcaaagaagccaaatacg |
| | | acatggcggtgggtcatctc |
| | | ttgctaaagtcattttgggc |
| | | gaatgaagccgtgtttcaaa |
| | | tgatgatgctttcatataac |
| | | ctatttttgttgttcaagtt |
| | | tgattccttggactcttcag |
| | | aatacagacagcaaataaag |
| | | acctttcgtttgaagtatgt |
| | | atttcttgcagcaaaaataa |
| | | tcaaaaccgcaagatatgta |
| | | atcatgaagttgtcggaaaa |
| | | ctatccgtacaagggagtgt |
| | | atgaaaaatgtctggtataa |
| | | taagaatatcatcaataaaa |
| | | ttgagtgttgctctgtggat |
| | | aacttgcagagtttattaag |
| | | tatcattgcagcaaagatga |
| | | aatcaatgatttatcaaaaa |
| | | tgattgaaaggtggttgtaa |
| | | ataatgttacaatgtgtgag |
| | | aagcagtctaaattcttcgt |
| | | gaaatagtgatttttgaagc |
| | | taataaaaaacacacgtgga |
| | | atttagggactattcatgtt |
| | | gttgttatttcgtatcttcc |
| | | agaataaggaatcccatggt |
| | | taaaaaatcactgcgccagt |
| | | tcacgctgatggcgacggca |
| | | accgtcacgctgttgttagg |
| | | aagtgtgccgctgtatgcgc |
| | | aaacggcggacgtacagcaa |
| | | aaacttgccgaattagagcg |
| | | gcagtcgggaggcagactgg |
| | | gtgtggcattgattaacaca |
| | | gcagataattcgcaaatact |
| | | ttatcgtgctgatgagcgct |
| | | ttgcgatgtgcagcaccagt |
| | | aaagtgatggccgcggccgc |
| | | ggtgctgaagaaaagtgaaa |
| | | gcgaaccgaatctgttaaat |
| | | cagcgagttgagatcaaaaa |
| | | atctgaccttgttaactata |
| | | atccgattgcggaaaagcac |
| | | gtcaatgggacgatgtcact |
| | | ggctgagcttagcgcggccg |
| | | cgctacagtacagcgataac |
| | | gtggcgatgaataagctgat |
| | | tgctcacgttggcggcccgg |
| | | ctagcgtcaccgcgttcgcc |
| | | cgacagctgggagacgaaac |
| | | gttccgtctcgaccgtaccg |
| | | agccgacgttaaacaccgcc |
| | | attccgggcgatccgcgtga |
| | | taccacttcacctcgggcaa |
| | | tggcgcaaactctgcggaat |
| | | ctgacgctgggtaaagcatt |
| | | gggcgacagccaacgggcgc |
| | | agctggtgacatggatgaaa |
| | | ggcaataccaccggtgcagc |
| | | gagcattcaggctggactgc |
| | | ctgcttcctgggttgtgggg |
| | | gataaaaccggcagcggtgg |
| | | ctatggcaccaccaacgata |
| | | tcgcggtgatctggccaaaa |
| | | gatcgtgcgccgctgattct |
| | | ggtcacttacttcacccagc |
| | | ctcaacctaaggcagaaagc |
| | | cgtcgcgatgtattagcgtc |
| | | ggcggctaaaatcgtcaccg |
| | | acggtttgtaatagg |
| 8 | nucleic acid | Catgttgttgttatttcgtat |
| | sequence of the | cttccagaataaggaatccc |
| | Eschehchia coli | atggttaaaaaatcactgcg |
| | bla_{CTX-M-15} gene | ccagttcacgctgatggcga |
| | by the 41 | cggcaaccgtcacgctgttg |
| | bases of the | ttaggaagtgtgccgctgta |
| | associated | tgcgcaaacggcggacgtac |
| | upstream | agcaaaaacttgccgaatta |
| | insertional | gagcggcagtcgggaggcag |
| | element ISEcp1 | actgggtgtggcattgatta |
| | directly | acacagcagataattcgcaa |
| | preceding the | atactttatcgtgctgatga |
| | bla_{CTX-M-15} | gcgctttgcgatgtgcagca |
| | gene | ccagtaaagtgatggccgcg |
| | | gccgcggtgctgaagaaaag |
| | | tgaaagcgaaccgaatctgt |
| | | taaatcagcgagttgagatc |
| | | aaaaaatctgaccttgttaa |
| | | ctataatccgattgcggaaa |
| | | agcacgtcaatgggacgatg |
| | | tcactggctgagcttagcgc |
| | | ggccgcgctacagtacagcg |
| | | ataacgtggcgatgaataag |
| | | ctgattgctcacgttggcgg |
| | | cccggctagcgtcaccgcgt |
| | | tcgcccgacagctgggagac |
| | | gaaacgttccgtctcgaccg |
| | | taccgagccgacgttaaaca |
| | | ccgccattccgggcgatccg |
| | | cgtgataccacttcacctcg |
| | | ggcaatggcgcaaactctgc |
| | | ggaatctgacgctgggtaaa |
| | | gcattgggcgacagccaacg |
| | | ggcgcagctggtgacatgga |
| | | tgaaaggcaataccaccggt |
| | | gcagcgagcattcaggctgg |
| | | actgcctgcttcctgggttg |
| | | tgggggataaaaccggcagc |
| | | ggtggctatggcaccaccaa |
| | | cgatatcgcggtgatctggc |
| | | caaaagatcgtgcgccgctg |
| | | attctggtcacttacttcac |
| | | ccagcctcaacctaaggcag |
| | | aaagccgtcgcgatgtatta |
| | | gcgtcggcggctaaaatcgt |
| | | caccgacggtttgtaa |
| 9 | LON_{control} | TTA GTT GGG GTT CAG |
| | | TTG G |
| 10 | Nucleotide | CGGCACACTTCCTAACAACA |
| | sequence of | |
| | the LASO_{ϵ} | |
| | antisense | |
| | oligonucleotide | |
| 11 | Nucleotide | ACGGTCGAGACGGAACGTTT |
| | sequence of | |
| | the LASO_{ζ} | |
| | antisense | |
| | oligonucleotide | |
| 12 | Nucleotide | AGGCTGGGTGAAGTAAGTGA |
| | sequence of | |
| | the LASO_{η} | |
| | antisense | |
| | oligonucleotide | |
| 13 | Nucleotide | GCGCAGTGATTTTTTAACCA |
| | sequence of | TGGGA |
| | the gapmer | |
| | LNA-5′LASOα | |
| | and of the | |
| | gapmer | |
| | LNA-ASOα | |
| | antisense | |
| | oligonucleotides | |

### BRIEF DESCRIPTION OF THE FIGURES

FIG. 1: Effect of lipid-modified antisense oligonucleotides on ceftriaxone MIC and viability in sensitive E. coli K12 strain after 24 h of incubation.

FIG. 2: Effect of lipid-modified antisense oligonucleotides on ceftriaxone MIC and viability in resistant laboratory (TcK12) and resistant clinical strain Ec3536.

FIG. 3: Effect of lipid-modified antisense oligonucleotide (LASO) with the lipid modification in 3′ or 5′ position compared to non-conjugated antisense oligonucleotides (ASO) on the MIC of E. coli sensitive K12 strain.

FIG. 4: Effect of LASO (modified either at the 5′ or 3′ extremities) on the ceftriaxone MIC after 24 h of incubation on laboratory resistant TcK12 strain.

FIG. 5: Effect of LASO (modified either at the 5′ or 3′ extremities) on the ceftriaxone MIC after 24 h of incubation on clinical resistant Ec3536 strain.

FIG. 6: β-lactamase quantification in E. coli TcK12 in presence of LASO_{α} (as a % of LON_{control}) using a colorimetric dosage.

FIG. 7: Effect of lipid derivatives of LNA-PTO gapmers on the ceftriaxone MIC on laboratory resistant TcK12 strain.

### EXAMPLES

### Example 1

This example describes the series of antisense oligonucleotide (ASO) sequences designed by the inventors targeting the bla_{CTX-M-15} gene featuring a lipid moiety conjugated to the ASO extremity to improve their intracellular penetration in prokaryotic cells and a phosphorothioate chemistry (PTO) for enzymatic stability.

### Materials and Methods

### Strains and Materials

E. coli strains used included the clinical strain Ec3536 collected from a urine sample of the community patient and provided from the MFP Laboratory collection (Arpin et al. (2009) J. Antimicrob. Chemother. 63:1205-1214). Its conjugative plasmid containing the bla_{CTX-M-15} gene was transferred by conjugation experiment in a laboratory recipient cell of E. coli K12. Consequently, the transconjugant E. coli TcK12 was resistant to ceftriaxone (CFX) (Arpin et al. (2009) J. Antimicrob. Chemother. 63:1205-1214).

Mueller-Hinton bacteria culture medium adjusted in calcium and magnesium ions (MH-CA) and microbiology consumable were purchased from Bio-Rad, France.

Ceftriaxone heptahemihydrate di-sodium salt was from Discovery Fine Chemical (UK), pharmaceutical grade, batch number: 74786.

Methanol and Acetonitrile (HPLC grade) were purchased from VWR (France).

Demineralized water was prepared at the laboratory by ion exchange (Pure Lab Option ELGA) followed by distillation (Water Still Distinction D4000).

### Synthesis, Purification and Dosage of Antisense Oligonucleotides (ASOs) and Lipid Conjugated Antisense Oligonucleotides (LASOs)

The ASOs/LASOs synthesis was performed on an automated Expedite 8909 DNA synthesizer at the μmol scale on 1000 Å primer support (loading: 30-100 μmol/g, Link technologies, Synbase Control Pore Glass). The cycled synthesis consisted of 4 steps: detritylation, coupling, oxidation and capping. The coupling of a double-chain nucleolipid (ketal-bis-C₁₅-Uridine) was performed by the Phosphoramidite methodology at the 5′ end of PTO-ASOs.

### Oligonucleotides Purification

### Chromatographic Analysis of Purity

All oligonucleotides synthesized were analysed by using High Performance Liquid Chromatography (HPLC) on Elite LaChrom (VWR) system with a Diode detector at 260 nm and injection volume of 20 μl during 15 min.

More particularly, for ASOs, hydrophobic column Xbridge oligonucleotide BEH C₁₈ (Waters) with particles' size of 2.5 μm, 130 Å of porosity and 4.6×50 mm of geometry was used. The mobile phase with 2.8 ml/min flow used was 70% of 95% of triethyl ammonium acetate (TEAA) at 100 mM+5% of Acetonitrile (ACN) at pH 7 and 30% of 20% of TEAA 20 mM and 80% of ACN.

For LASOs, Nucleosil C₄ column with 4×250 mm geometry and particles size of 5 μm, 300 Å of porosity (Macherey Nagel) was used with 1.0 ml/min of 20% of TEAA 20 mM and 80% of CAN as mobile phase.

Oligonucleotides purification for LASO was performed using preparative HPLC method with column XBridge Protein BEH C₄ OBD Pre with 30×50 mm of geometry, particles size of 5 μM and porosity of 300 Å. The mobile phase used was 20% of TEAA 20 mM and 80% of CAN at 56.25 mL/min flow. The run of analysis was 4 min.

### Dialysis Method

A dialysis system was used to desalt the purified samples. The columns of Vivaspin Turbo 4 (Sartorius, cut-off 3.5 kDa, membrane Polyethersulfone) were used for oligonucleotides desalting.

Membranes were rinsed with distilled water and then samples were added into the column before being centrifuged at 3000 rpm for 30 min. Three washings were made by adding 2 mL of distilled water into the superior part of the tube and then re-centrifuged as previously. 500 μL of distilled water was added on the membrane to re-suspend oligonucleotide and collect it. Then the membrane was rinsed 3 times with 500 μL of distilled water.

### ASOs/LASOs Assay

The concentration of all ASOs and LASOs was determined by spectrophotometry Nanodrop© (Thermo Scientific™) at 260 nm with automatic oligonucleotide detection mode.

### Dynamic Light Scattering (DLS) Characterization

The size of LASOs objects was measured at room temperature using Zetasizer Nano ZS90 (Malvern Instruments Ltd., UK). Size was measured in a specific cell ZEN 0040 (Malvern, France) for NPs and Zeta Potential in a DTS 1070 cell (Malvern, France). Measurement conditions were: material Protein (RI: 1.450; Absorption: 0.001), dispersant water (Viscosity: 0.8872 cP; RI: 1.330) temperature at 25° C. or 37° C. and equilibration time was 120 s. Each test was triplicated.

### Effect of ASOs/LASOs on the Sensitivity of Bacteria to CFX

Determination of minimum inhibitor concentration (MICs) of free CFX with or without ASOs/LASOs was performed on different strains of E. coli, i.e. the CFX sensitive strain K12, and the two resistant strains TcK12 and Ec3536.

### General Procedure of MICs Determination

MICs of free CFX with ASOs/LASOs were determined in accordance with the standard method of liquid micro-dilution.

Each bacterial strain that had been frozen at −80° C. was isolated on Mueller-Hinton (MH) agar during 16 h at 37° C. A bacterial suspension in solution of 0.85% NaCl was prepared in order to obtain a turbidity equivalent to standard 0.5 of McFarland range and then diluted 1/100 in MH that correspond to a bacterial inoculum of ca. 10⁶ CFU/mL. The bacterial suspension was afterwards mixed with ASOs, LASOs at 2-fold desired concentration in MH. Then 50 μL of bacterial suspension mixed with 50 μl MH, containing oligonucleotides or CFX, was dispensed into the microplate wells immediately. The final volume per well was 100 μL. The concentration of oligonucleotides in well was fixed at 5 μM except for dose-effect tests. The concentration range of LASOs from 0.05 μM to 50 μM was tested. The range of CFX concentration was adjusted to surround the MIC of each bacterial strain. Microplates were incubated at 35±2° C. for 24 h.

Preliminary data allowed to optimize the analysis of microplates using turbidimeter (Apollo LB 911 (Berthold)) at the wavelength of 620 nm, read in triplicate at 0 h, and 24 h. The blank optical density (TO) was subtracted to 24 h results. The MIC was determined as being the lowest concentration of CFX where no turbidity was observed with the optical density (abs<0.05). For unclear cases, MTT test (Cell Titer 96@ Aqueous One Solution Cell Proliferation Assay, Promega, USA) was used to confirm the bacterial growth. All tests for MICs determination were triplicated in independent tests.

Determination of 3-Lactamase Inhibition with Colorimetric Method.

β-lactamase activity was measured by hydrolyzing of the nitrocefin, a chromogenic cephalosporin. Nitrocefin degradation led to a colorimetric product proportional to the enzymatic activity.

An inoculum corresponding to 10⁶ CFU/mL of E. coli TcK12 in presence of LASO_{α} or LON_{control} was incubated at 37° C. during 5 h. Then, 10 μL of nitrocefin solution (50 mg/L, Thermo Scientific Oxoid™) was added into each well of the microplate and incubated at room temperature for 45 min before reading by using turbidimeter (Apollo LB 911 (Berthold)) at the wavelength of 492 nm. Each measure was made in triplicate. The blank optical density was subtracted to value without incubation with nitrocefin.

### Optical Fluorescence Imaging of LASOs Localization in Bacterial Cells

ASO_{α-Cy5}/LASO_{α-Cy5} at the concentration of 5 μM incubated with 5×10⁴ CFU/ml of TcK12 strain during 20 h were observed under confocal fluorescent microscopy with 630× magnification and PMT4 Detector. Laser excitation's wavelength was 638 nm.

### Results

### Synthesis of Antisense Oligonucleotides (ASO) and Lipid-Modified Antisense Oligonucleotides (LASO)

All the oligonucleotide based derivatives used in this study were synthesized and characterized.

The oligonucleotide sequences used were chosen ASO/LASO according to literature (Readman et al. (2016) Front. Microbiol. 7:373) and in house developed sequences along with negative controls were synthetized with PTO backbone (Table 1).

TABLE 1
Sequences of tested ASOs and LASOs

| Name ^{(a)} | Length (mers) | Sequence (5′→3′) |
|---|---|---|
| (L)ASO_{α} ^{(b)} | 25 | GCG CAG TGA TTT TTT |
| | | AAC CAT GGG A |
| | | (SEQ ID NO: 1) |
| (L)ASO_{β} | 19 | CGT GTA GGT ACG GCA |
| | | GAT C |
| | | (SEQ ID NO: 2) |
| (L)ASO_{γ} | 25 | TGA ACT GGC GCA GTG |
| | | ATT TTT TAA C |
| | | (SEQ ID NO: 3) |
| (L)ASO_{δ} | 21 | GTC GGC TCG GTA CGG |
| | | TCG AGA |
| | | (SEQ ID NO: 4) |
| LON_{control} | 19 | TTA GTT GGG GTT CAG |
| | | TTG G (SEQ ID NO: 9) |

^{(a)} LASOs being 5′ or 3′ conjuguates of ASO with ketal bis-C₁₅ lipid
^{(b)} Cyanine 5 was coupled to the 3′ end of 5′(L)ASO_{α}

Briefly, the oligonucleotides were modified at the 5′-end with different lipid phosphoramidites. The phosphoramidites single chain 1 and 2 were synthesized according to literature procedures and coupled to the 5′-end of the oligonucleotides (Gissot et al. (2008) Chem. Commun. 43:5550-5552). “Scramble” oligonucleotide sequences were also synthesized as controls wherein the sequence do not target undesired mRNA sequences.

### Physicochemical Characterization of Micelles.

While ASOs remain in aqueous solution without specific self-organization (no significant population of objects in water by DLS), LASOs organize themselves in micelles and larger objects. The mean size of micellar population of different sequences measured by Dynamic Light Scattering (DLS) in extracellular salt conditions (145 mM Na⁺ and 5 mM K⁺) ranged around 10 nm (Table 2), independently of the oligonucleotide sequence, with negative zeta potential, as expected regarding polyanion structure of oligonucleotides.

TABLE 2
DLS (number mode) size of micellar population of
oligonucleotide assemblies at 30 μM, 25° C. in extracellular
salt conditions (145 mM NaCl and 5 mM KCl)

| Name of oligonucleotide | Length | Micellar size (nm, |
|---|---|---|
| sequence | (bases) | DLS number) |
| ^{5′}LASO_{α} | 25 | 11.6 +/− 0.8 |
| ^{5′}LASO_{β} | 19 | 10.2 +/− 0.4 |
| ^{5′}LASO_{γ} | 25 | 10.8 +/− 0.5 |
| ^{5′}LASO_{δ} | 21 | 6.5 +/− 0.6 |
| ^{5′}LON_{control} | 19 | 11.0 +/− 0.2 |

The size was shown to be independent upon LASO concentration and room vs physiological temperature.

### Bacterial Viability.

The effect of antisense sequences as well as their lipid conjugates was studied on two Escherichia coli laboratory strains: the sensitive strain K12 and its resistant transconjugant TcK12 which contains a conjugative plasmid with the bla_{CTX-M-15} gene. The effect of antisense sequences was further confirmed on the clinical E. coli strain, Ec3536 (Arpin et al. (2009) J. Antimicrob. Chemother. 63:1205-1214).

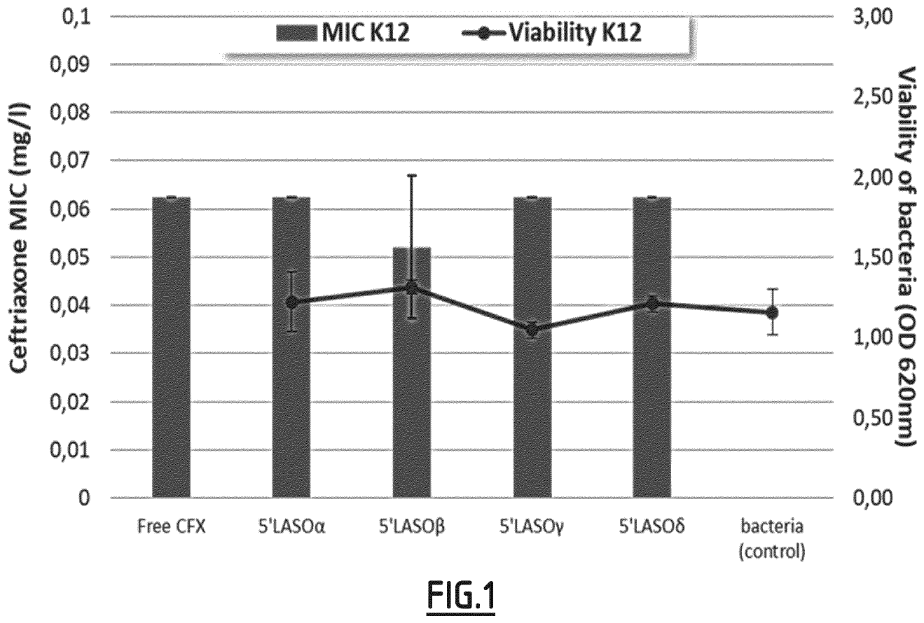

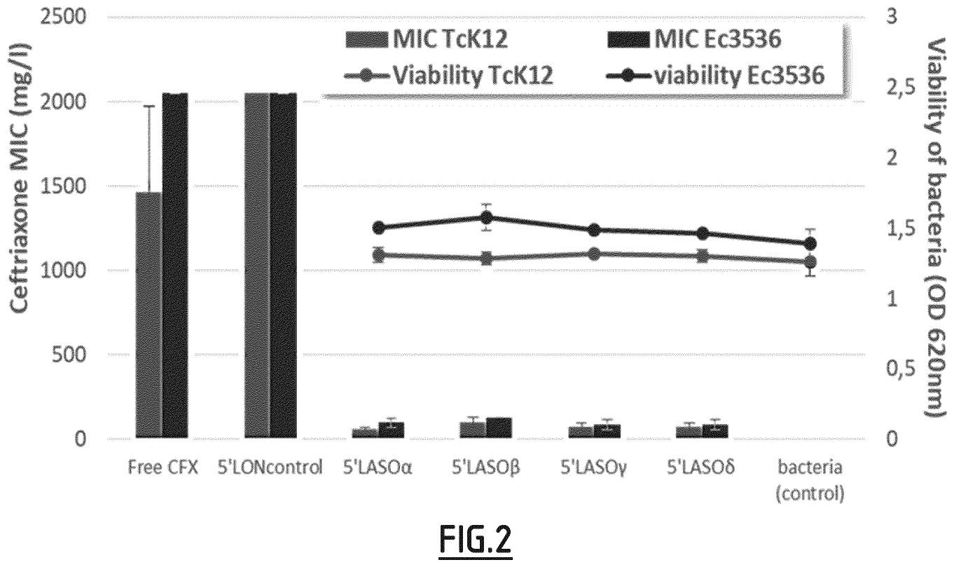

The results showed that the presence of sequences with lipid conjugates did not affect bacterial viability (FIGS. 1 and 2 right axis, p>0.48).

### Effect on the Sensitivity of Bacteria to CFX

When tested on sensitive laboratory strain E. coli K12, the MIC found in absence of antisense sequences was 0.06 mg/L (SD 0, n=3) of CFX (FIG. 1, left axis). The presence of neither antisense sequences nor their lipid conjugates affected the MIC significantly (FIG. 1, left axis).

The effect of antisense sequences and lipid conjugates was further tested on the resistant laboratory strain, TcK12. The results (FIG. 2, left axis) show an important decrease of CFX MIC in presence of LASOs.

Among sequences reported in literature, in particular in Readman et al. (2016) Front Microbiol. 7:373 and Readman et al. (2017) Nucleic Acids Ther. 27:176, the corresponding PTO sequence of ^{5′} LASOₐ (concentration of 5 μM) was the most potent lipid conjugate for CFX MIC decrease on resistant E. coli TcK12 strain, with a 26-fold decrease (means of MICs, 56 mg/L with ^{5′} LASO_{α} vs 1365 mg/L without ^{5′} LASO_{α}, FIG. 2). As observed on sensitive K12 strain, no effect on MIC nor on bacterial viability was observed (FIG. 1).

No CFX MIC decrease was obtained with the 5′LON_{control}, tested in the same conditions (FIG. 2). These results of CFX MIC decrease were confirmed on the resistant clinical strain of E coli Ec3536 (FIG. 2).

The effect of LASOs on MIC was further shown to be dose-dependent. The concentration of 5 μM chosen for the initial screening corresponded to the minimal concentration to reach the minimum MIC.

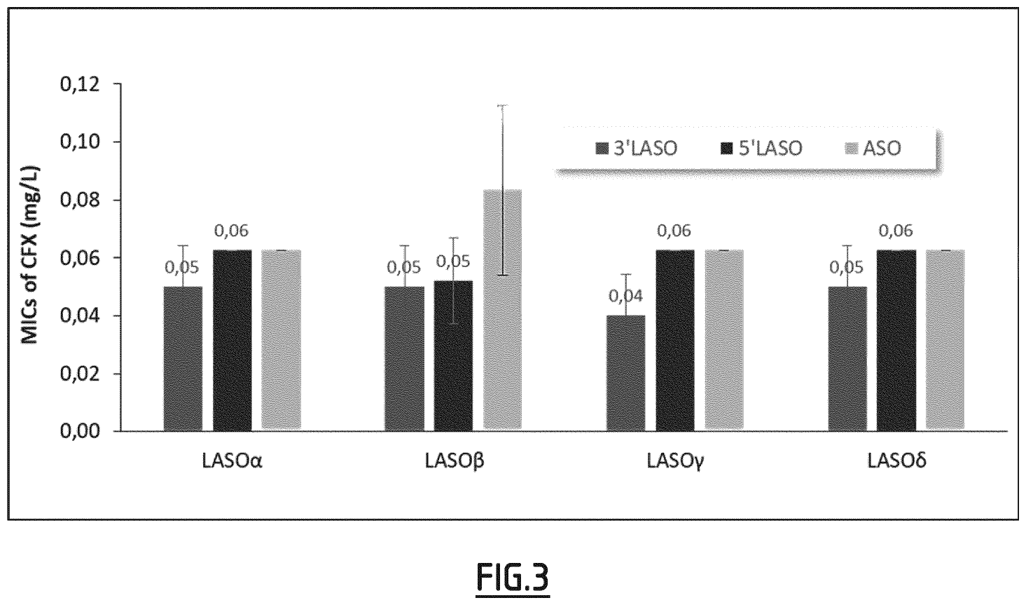

The position of the lipid, initially inserted at the 5′ oligonucleotide extremity via a 5′-5′ linkage was modified to 3′ position. The results showed that while sensitive E. coli strains were not affected (FIG. 3), resistance of bacteria was partially reversed with 3′ lipid conjugates.

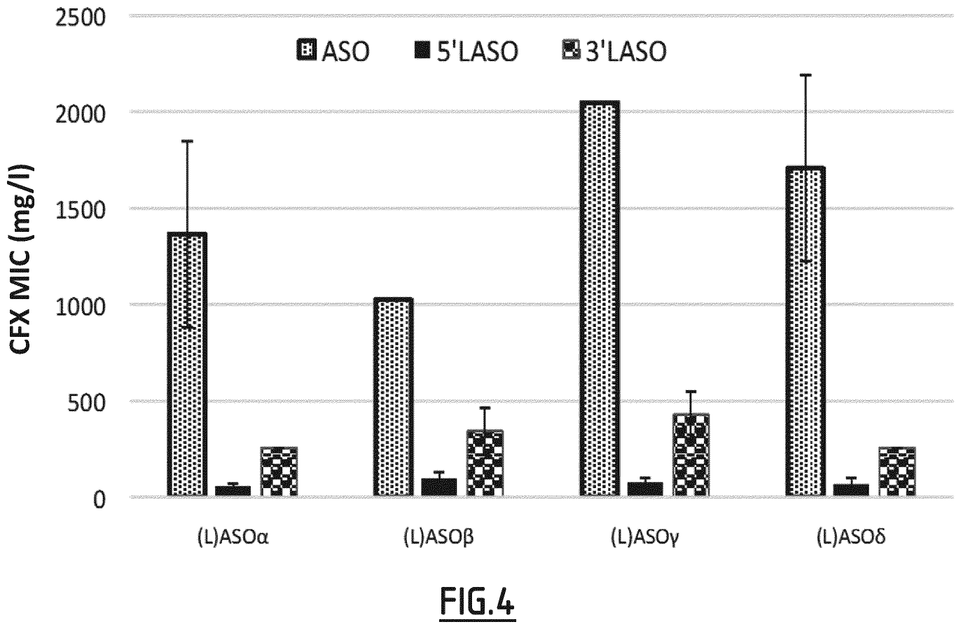

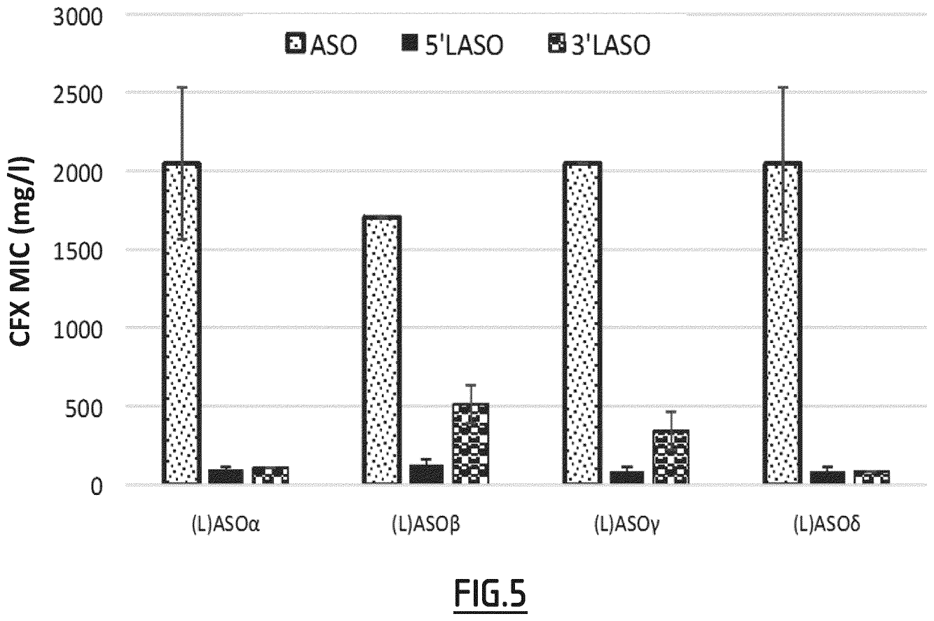

The result was sequence-dependent and strain dependent (FIGS. 4 and 5 for clinical and laboratory resistant strains respectively).

### Intra-Bacterial Penetration

In order to demonstrate LASO intra-bacterial penetration and effect, Cyanine 5 was coupled to the 3′ extremity of ^{5′} LASO_{α} sequence.

While not affecting the MIC, the fluorescent microscopy allowed to visualize intra-bacterial localization of ^{5′} LASO_{α}. ASO (ASO_{α-Cy5}) resulted only in an enhanced background noise.

### Effect on β-Lactamase Activity

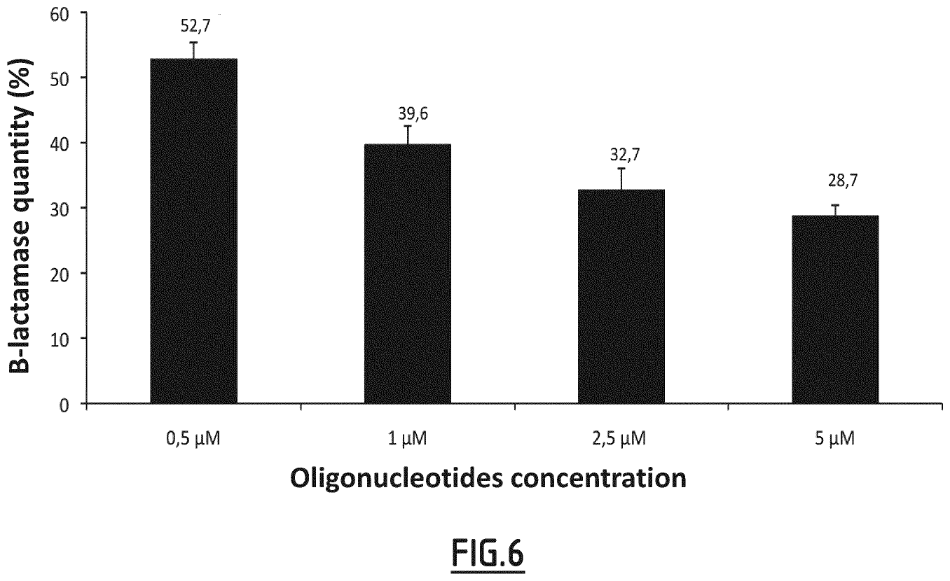

The β-lactamase quantity was investigated by using a chromogenic cephalosporin, the nitrocefin. An inhibition was observed in E. coli TcK12 cultivated in presence of different concentrations of LASO_{α} compared to LON_{control} (FIG. 6).

### Discussion

In the present study, the inventors generated lipid conjugates featuring antisense oligonucleotide sequences targeting β-lactamase mRNA in resistant bacteria. From the inventors' knowledge, such a lipid modification has not been investigated in the context of delivering nucleic acids into prokaryotic cells, and especially in Gram-negative bacteria which possess in their bacterial cell wall both peptidoglycan and outer membrane. The aim of this study was to tackle the antibiotic resistance issue. To validate the inventors' approach, a family of oligonucleotide conjugates was investigated with ceftriaxone as a β-lactam antibiotic.

β-lactam, including penicillins, cephalosporins, carbapenems and monobactam are the most used antibiotics for the treatment of bacterial infections. The main targets of these drugs are penicillin-binding proteins (PBPs). It is well documented that the interactions between the β-lactam ring and PBP results in an inhibition of the cell wall's peptidoglycans synthesis, which induces the bacterial lysis.

The ceftriaxone (CFX), used in this study, is a broad-spectrum antibiotic, which belongs to 3r generation cephalosporins. This antibiotic was selected because it is one of the most commonly used antibiotics due to its high antibacterial efficacy, wide spectrum of activity, prolonged half-life allowing once a day dosing and low potential for toxicity. Its widespread use can be explained by its effectiveness in susceptible microorganisms infections of urinary tract, respiratory tract, skin, soft tissue, bone and joint. Also it has been used against infections in immunosuppressed patients, acute bacterial otitis media, genital infections, disseminated Lyme's disease, bacteremia/septicemia, meningitis, and in surgical prophylaxis of infections.

Among the acquired mechanisms of CFX resistance, the production of ESBLs is one of the most common mechanism in enterobacteria. ESBLs' action mechanism is to cleave the amide bond in the β-lactam ring, resulting in an inactivation of β-lactam antibiotics. In this family, the group of CTX-M β-lactamases and specifically the type CTX-M-15 β-lactamase that are highly resistant to cefotaxime and CFX are the most frequent ESBLs at the worldwide level.

The aim of the present study was to propose a new approach based on antisense (ASO) targeting the mRNA sequences coding for the production of CTX-M-15 β-lactamase. In this context, a series of ASO featuring phosphorothiate (PTO) chemistry was modified with lipid moiety at either 3′ or 5′ extremities to increase the cellular uptake.

As outlined above, cellular uptake is an important feature for the ASO strategy, as the oligonucleotides have to reach the mRNA to inhibit the production of β-lactamase. Spherical micellar assemblies with average diameter ranging from 6.5 and 11.6 nm were observed spontaneously in aqueous media. These micelles would be responsible to the bacteria internalization as observed by confocal microscopy imaging of E. coli TcK12 incubated in the presence of LASO_{α-Cy5}. Importantly, only the lipid-modified oligonucleotides LASO were efficient in decreasing the MIC of ceftriaxone on two different resistant strains (TcK12 and clinical Ec3536), while the corresponding non-lipidic ASO did not show any impact on the MIC.

The specific antisense effect of lipid-ASO conjugates was confirmed by the absence of effect of non-binding lipid oligonucleotide (LON_{control}) on the MIC, suggesting that binding the mRNA sequence is responsible of the biological effect. Also, the LASO effect was found to be dose-dependent as revealed by the MIC study achieved on TcK12 at different LASO concentrations. A LASO concentration of 5 μM was found to be the optimal concentration.

The biological activity of LASO is correlated to its affinity for mRNA (inducing either a RNAse H dependent cleavage or a steric hindrance avoiding mRNA-ribosome interactions) leading in both cases to the inhibition of the translation of CTX-M-15 β-lactamase. This inhibition was showed by measuring its hydrolysis activity on a chromogenic cephalosporin in E. coli TcK12 cultivated in presence of LASO_{α} compared to LON_{control}, supporting a specific translational inhibition of the 3 lactamase by LASO.

The decrease of β lactamase was also dependent on the LASO concentration.

Finally, it was found that the non cytotoxic LASOs led to a strong decrease in MICs (more than 25 fold decrease). Such an effect using antisense approach on both clinical and laboratories resistant strains has been never reached before. The fast and remarkable killing of the resistant bacteria strains after LASOs treatments was explained by: i) an important intrabacteria capture and ii) the decrease of β-lactamase expression thanks to the oligonucleotide sequences targeting the bla_{CTX-M-15} gene.

Consequently, this example demonstrates the strong potential of the LASO strategy in restoring the antimicrobial activities of cephalosphorins against resistant bacteria. This approach, which can be adapted to other antimicrobial drugs, opens promising perspectives in the struggle against a worldwide public health issue such as the bacterial resistance.

### Example 2

TABLE 3
Sequences of antisense oligonucleotides

| SEQ | | |
|---|---|---|
| ID | Description | Sequence |
| 10 | Nucleotide sequence | CGGCACACTT |
| | of the LASO_{ϵ} antisense | CCTAACAACA |
| | oligonucleotide, as | |
| | an example of the lipid | |
| | modified sequence | |
| | targeting gene region | |
| | from base 53 to 75 | |
| | counting from ATG | |
| 11 | Nucleotide sequence | ACGGTCGAGA |
| | of the LASO_{ζ} antisense | CGGAACGTTT |
| | oligonucleotide, as an | |
| | example of the lipid | |
| | modified sequence | |
| | targeting gene region | |
| | from base 480 to 500 | |
| | counting from ATG | |
| 12 | Nucleotide sequence | AGGCTGGGTG |
| | of the LASO_{η} | AAGTAAGTGA |
| | antisense oligonucleotide | |
| | as an example of | |
| | the lipid modified | |
| | sequence targeting | |
| | gene region from base | |
| | 781 to 805 counting | |
| | from ATG | |

The inventors evaluated the effect of these antisense oligonucleotides on ceftriaxone MIC in E. coli TcK12 strain at 5 μM after 24 h. The results obtained are displayed in Table 4 below.

TABLE 4
Effect of antisense oligonucleotides on ceftriaxone
MIC in E. coli TcK12 strain

| Description | MIC ASO/MIC LASO (mg/L) |
|---|---|
| LASO_{ε} | 1024/64 |
| LASO_{ζ} | 1024/64 |
| LASO_{η} | 1024/512 |
| control | 1024 |

The inventors thus showed that the antisense oligonucleotides of the invention decreased MIC of ceftriaxone in E. coli resistant TcK12 resistant strains.

### Example 3

The inventors further evaluated the effect of gapmers LNA PTO chemically modified or not with a lipid conjugate.

The synthesis of additional chemical modifications with or without lipid conjugate, i.e. LNA gapmer and negatively charged morpholino oligonucleotides was performed.

The 4-16-4 LNA PTO gapmers used classical syntheses pathway in 3′-5′ direction, as described for PTO oligonucleotides. Negatively charged morpholino phosphodiester were successfully synthetized from phophoramidite morpholino monomers from Sapala Organics Private Limited in 5′-3′ direction.

The LNA gapmers synthesized were as follows:
Gapmer LNA LASOα (chemically modified with lipid conjugate)

(SEQ ID NO: 13)
5′-(NL) GCG CAG TGA TTT TTT AAC CAT GGG A-3′

Underlined: LNA (PTO)
Not underlined: classical PTO
Gapmer LNA ASOα (without lipid modification)

(SEQ ID NO: 13)
5′-GCG CAG TGA TTT TTT AAC CAT GGG A-3′

Underlined: LNA (PTO)
Not underlined: classical PTO

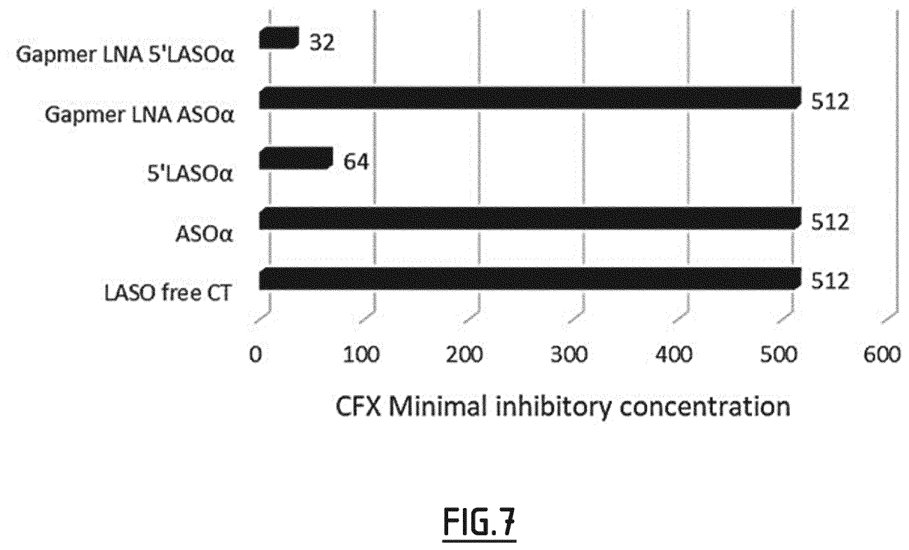

Compared to PTO oligonucleotides, lipid derivatives of Locked nucleic acid (LNA)-PTO gapmer showed equivalent efficiency in reducing ceftriaxone MIC down to 32 mg/ml, whereas the non-lipid oligonucleotide gapmer remained unchanged compared to control (see FIG. 7).

1. Antisense oligonucleotide modified by substitution at the 5′ or the 3′ end by a lipid moiety, wherein said antisense oligonucleotide specifically targets an mRNA encoding a CTX-M extended spectrum β-lactamase.

2. The antisense oligonucleotide according to claim 1, wherein the lipid moiety is a moiety comprising at least one ketal functional group, wherein the ketal carbon of said at least one ketal functional group bears two saturated or unsaturated, linear or branched, hydrocarbon chains comprising from 1 to 22 carbon atoms.

3. The antisense oligonucleotide according to claim 1, of the general formula (I) 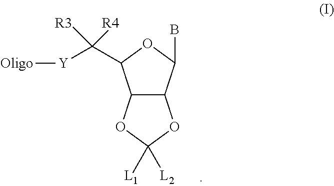
wherein:
Oligo represents an antisense oligonucleotide sequence specifically targeting an mRNA encoding a CTX-M extended spectrum β-lactamase, wherein said antisense oligonucleotide is optionally: oriented 3′-5′ or 5′-3′, single stranded, DNA, RNA, and/or comprises modified nucleotides;
Y represents a divalent linker moiety selected from the group consisting of —O—, thio —S—, amino —NH—, and methylene —CH₂—;
R₃ and R₄ may be identical or different and represent:
a hydrogen atom,
a halogen atom,
a hydroxyl group, or
an alkyl group comprising from 1 to 12 carbon atoms;
L₁ and L₂ may be identical or different and represent a saturated or unsaturated, linear or branched hydrocarbon chain comprising from 1 to 22 carbon atoms;
B is an optionally substituted nucleobase, selected from the group consisting of purine nucleobases, pyrimidine nucleobases, and non-natural monocyclic or bicyclic heterocyclic nucleobases wherein each cycle comprises from 4 to 7 atoms.

4. The antisense oligonucleotide according to claim 3, wherein
the divalent linker moiety is —O—,
R₁ and R₂ are hydrogen atoms,
L₁ and L₂ represent a hydrocarbon chain comprising from 6 to 22 carbon atoms, and
B is a non substituted nucleobase selected from the group consisting of uracil, thymine, adenine, cytosine, 6-methoxypurine and hypoxanthine.

5. The antisense oligonucleotide according to claim 1, wherein said antisense oligonucleotide is a phosphorothioate derivative.

6. The antisense oligonucleotide according to claim 1, wherein said CTX-M extended spectrum β-lactamase is a group 1 CTX-M extended spectrum β-lactamase.

7. The antisense oligonucleotide according to claim 1, wherein said CTX-M extended spectrum β-lactamase is the CTX-M-15 extended spectrum β-lactamase.

8. The antisense oligonucleotide according to claim 1, wherein said antisense oligonucleotide is or comprises a sequence selected from the group consisting of the sequences GCGCAGTGATTTTTTAACCATGGGA (SEQ ID NO: 1), CGTGTAGGTACGGCAGATC (SEQ ID NO: 2), TGAACTGGCGCAGTGATTTTTTAAC (SEQ ID NO: 3), GTCGGCTCGGTACGGTCGAGA (SEQ ID NO: 4), CGGCACACTTCCTAACAACA (SEQ ID NO: 10), ACGGTCGAGACGGAACGTTT (SEQ ID NO: 11) and AGGCTGGGTGAAGTAAGTGA (SEQ ID NO: 12).

9. Method of treating a bacterial infection in a subject in need thereof, comprising, administering to the subject a therapeutically effective amount of the antisense oligonucleotide according to claim 1.

10. The method according to claim 9, wherein said bacterial infection is an infection due to Enterobacteriaceae bacteria.

11. The method according to claim 9, wherein the antisense oligonucleotide is administered in combination with a 3^{rd} generation cephalosporin, 4^{th} generation cephalosporin and/or monobactam.

12. Pharmaceutical composition comprising (i) an antisense oligonucleotide as defined in claim 1, and (ii) a 3^{rd} generation cephalosporin, 4^{th} generation cephalosporin and/or monobactam.

13. (canceled)

14. Kit comprising:
(i) an antisense oligonucleotide as defined in claim 1, and
(ii) a 3^{rd} generation cephalosporin, 4^{th} generation cephalosporin and/or monobactam.

15-16. (canceled)

17. The antisense oligonucleotide of claim 3, wherein the halogen atom is fluorine.

18. The antisense oligonucleotide of claim 4, wherein L₁ and L₂ represent a hydrocarbon chain comprising from 8 to 18 carbon atoms or from 12 to 16 carbon atoms.

19. The method of claim 9, wherein the bacterial infection, is due to bacteria resistant to 3^{rd} generation cephalosporins, 4^{th} generation cephalosporins and/or monobactams.

20. The method according to claim 10, wherein the Enterobacteriaceae bacteria is a species of Escherichia coli.


SEQUENCE LISTING (13 sequences)

SEQ ID NO: 1 - DNA, 25 bp, Artificial Sequence
  Synthetic Nucleotide sequence of the LASOalpha antisense oligonucleotide
gcgcagtgattttttaaccatggga

SEQ ID NO: 2 - DNA, 19 bp, Artificial Sequence
  Synthetic Nucleotide sequence of the LASObeta antisense oligonucleotide
cgtgtaggtacggcagatc

SEQ ID NO: 3 - DNA, 25 bp, Artificial Sequence
  Synthetic Nucleotide sequence of the LASOgamma antisense oligonucleotide
tgaactggcgcagtgattttttaac

SEQ ID NO: 4 - DNA, 21 bp, Artificial Sequence
  Synthetic Nucleotide sequence of the LASOdelta antisense oligonucleotide
gtcggctcggtacggtcgaga

SEQ ID NO: 5 - DNA, 876 bp, Escherichia coli
atggttaaaaaatcactgcgccagttcacgctgatggcgacggcaaccgtcacgctgttgttaggaagtgtgccgctgtatgcgcaaacggcggacgtacagcaaaaacttgccgaattagagcggcagtcgggaggcagactgggtgtggcattgattaacacagcagataattcgcaaatactttatcgtgctgatgagcgctttgcgatgtgcagcaccagtaaagtgatggccgcggccgcggtgctgaagaaaagtgaaagcgaaccgaatctgttaaatcagcgagttgagatcaaaaaatctgaccttgttaactataatccgattgcggaaaagcacgtcaatgggacgatgtcactggctgagcttagcgcggccgcgctacagtacagcgataacgtggcgatgaataagctgattgctcacgttggcggcccggctagcgtcaccgcgttcgcccgacagctgggagacgaaacgttccgtctcgaccgtaccgagccgacgttaaacaccgccattccgggcgatccgcgtgataccacttcacctcgggcaatggcgcaaactctgcggaatctgacgctgggtaaagcattgggcgacagccaacgggcgcagctggtgacatggatgaaaggcaataccaccggtgcagcgagcattcaggctggactgcctgcttcctgggttgtgggggataaaaccggcagcggtggctatggcaccaccaacgatatcgcggtgatctggccaaaagatcgtgcgccgctgattctggtcacttacttcacccagcctcaacctaaggcagaaagccgtcgcgatgtattagcgtcggcggctaaaatcgtcaccgacggtttgtaa

SEQ ID NO: 6 - protein, 291 aa, Escherichia coli
MVKKSLRQFTLMATATVTLLLGSVPLYAQTADVQQKLAELERQSGGRLGVALINTADNSQILYRADERFAMCSTSKVMAAAAVLKKSESEPNLLNQRVEIKKSDLVNYNPIAEKHVNGTMSLAELSAAALQYSDNVAMNKLIAHVGGPASVTAFARQLGDETFRLDRTEPTLNTAIPGDPRDTTSPRAMAQTLRNLTLGKALGDSQRAQLVTWMKGNTTGAASIQAGLPASWVVGDKTGSGGYGTTNDIAVIWPKDRAPLILVTYFTQPQPKAESRRDVLASAAKIVTDGL

SEQ ID NO: 7 - DNA, 2315 bp, Artificial Sequence
  Synthetic nucleic acid sequence of the Escherichia coli blaCTX-M-15 gene preceded by the associated upstream insertional element ISEcp1
tgggatttttgattttattgaaaatgacctcgtatttgataatgactcaacaaataaaatcaagatgaatcatataaagaccatgctctgcggtcacttcattggcattgataagttagaacgtctaaagctacttcaaaatgatcccctcgtcaacgagtttgatatttccgtaaaagaacctgaaacagtgtcacggtttctaggaaacttcaacttcaagacaacccaaatgtttagagacattaattttaaagtctttaaaaaactgctcactaaaagtaaattgacatccattacgattgatattgatagtagtgtaattaacgtagaaggtcatcaagaaggtgcgtcaaaaggatataatcctaagaaactgggaaaccgatgctacaatatccaatttgcattttgcgacgaattaaaagcatatgttaccggatttgtaagaagtggcaatacttacactgcaaacggtgctgcggaaatgatcaaagaaattgttgctaacatcaaatcagacgatttagaaattttatttcgaatggatagtggctactttgatgaaaaaattatcgaaacgatagaatctcttggatgcaaatatttaattaaagccaaaagttattctacactcacctcacaagcaacgaattcatcaattgtattcgttaaaggagaagaaggtagagaaactacagaactgtatacaaaattagttaaatgggaaaaagacagaagatttgtcgtatctcgcgtactgaaaccagaaaaagaaagagcacaattatcacttttagaaggttccgaatacgactactttttctttgtaacaaatactaccttgctttctgaaaaagtagttatatactatgaaaagcgtggtaatgctgaaaactatatcaaagaagccaaatacgacatggcggtgggtcatctcttgctaaagtcattttgggcgaatgaagccgtgtttcaaatgatgatgctttcatataacctatttttgttgttcaagtttgattccttggactcttcagaatacagacagcaaataaagacctttcgtttgaagtatgtatttcttgcagcaaaaataatcaaaaccgcaagatatgtaatcatgaagttgtcggaaaactatccgtacaagggagtgtatgaaaaatgtctggtataataagaatatcatcaataaaattgagtgttgctctgtggataacttgcagagtttattaagtatcattgcagcaaagatgaaatcaatgatttatcaaaaatgattgaaaggtggttgtaaataatgttacaatgtgtgagaagcagtctaaattcttcgtgaaatagtgatttttgaagctaataaaaaacacacgtggaatttagggactattcatgttgttgttatttcgtatcttccagaataaggaatcccatggttaaaaaatcactgcgccagttcacgctgatggcgacggcaaccgtcacgctgttgttaggaagtgtgccgctgtatgcgcaaacggcggacgtacagcaaaaacttgccgaattagagcggcagtcgggaggcagactgggtgtggcattgattaacacagcagataattcgcaaatactttatcgtgctgatgagcgctttgcgatgtgcagcaccagtaaagtgatggccgcggccgcggtgctgaagaaaagtgaaagcgaaccgaatctgttaaatcagcgagttgagatcaaaaaatctgaccttgttaactataatccgattgcggaaaagcacgtcaatgggacgatgtcactggctgagcttagcgcggccgcgctacagtacagcgataacgtggcgatgaataagctgattgctcacgttggcggcccggctagcgtcaccgcgttcgcccgacagctgggagacgaaacgttccgtctcgaccgtaccgagccgacgttaaacaccgccattccgggcgatccgcgtgataccacttcacctcgggcaatggcgcaaactctgcggaatctgacgctgggtaaagcattgggcgacagccaacgggcgcagctggtgacatggatgaaaggcaataccaccggtgcagcgagcattcaggctggactgcctgcttcctgggttgtgggggataaaaccggcagcggtggctatggcaccaccaacgatatcgcggtgatctggccaaaagatcgtgcgccgctgattctggtcacttacttcacccagcctcaacctaaggcagaaagccgtcgcgatgtattagcgtcggcggctaaaatcgtcaccgacggtttgtaatagg

SEQ ID NO: 8 - DNA, 917 bp, Artificial Sequence
  Synthetic nucleic acid sequence of the Escherichia coli blaCTX-M-15 gene by the 41 bases of the associated upstream insertional element ISEcp1 directly preceding the blaCTX-M-15 gene
catgttgttgttatttcgtatcttccagaataaggaatcccatggttaaaaaatcactgcgccagttcacgctgatggcgacggcaaccgtcacgctgttgttaggaagtgtgccgctgtatgcgcaaacggcggacgtacagcaaaaacttgccgaattagagcggcagtcgggaggcagactgggtgtggcattgattaacacagcagataattcgcaaatactttatcgtgctgatgagcgctttgcgatgtgcagcaccagtaaagtgatggccgcggccgcggtgctgaagaaaagtgaaagcgaaccgaatctgttaaatcagcgagttgagatcaaaaaatctgaccttgttaactataatccgattgcggaaaagcacgtcaatgggacgatgtcactggctgagcttagcgcggccgcgctacagtacagcgataacgtggcgatgaataagctgattgctcacgttggcggcccggctagcgtcaccgcgttcgcccgacagctgggagacgaaacgttccgtctcgaccgtaccgagccgacgttaaacaccgccattccgggcgatccgcgtgataccacttcacctcgggcaatggcgcaaactctgcggaatctgacgctgggtaaagcattgggcgacagccaacgggcgcagctggtgacatggatgaaaggcaataccaccggtgcagcgagcattcaggctggactgcctgcttcctgggttgtgggggataaaaccggcagcggtggctatggcaccaccaacgatatcgcggtgatctggccaaaagatcgtgcgccgctgattctggtcacttacttcacccagcctcaacctaaggcagaaagccgtcgcgatgtattagcgtcggcggctaaaatcgtcaccgacggtttgtaa

SEQ ID NO: 9 - DNA, 19 bp, Artificial Sequence
  Synthetic LONcontrol
ttagttggggttcagttgg

SEQ ID NO: 10 - DNA, 20 bp, Artificial Sequence
  Synthetic Nucleotide sequence of the LASOepsilon antisense oligonucleotide
cggcacacttcctaacaaca

SEQ ID NO: 11 - DNA, 20 bp, Artificial Sequence
  Synthetic Nucleotide sequence of the LASOdzeta antisense oligonucleotide
acggtcgagacggaacgttt

SEQ ID NO: 12 - DNA, 20 bp, Artificial Sequence
  Synthetic Nucleotide sequence of the LASOeta antisense oligonucleotide
aggctgggtgaagtaagtga

SEQ ID NO: 13 - DNA, 25 bp, Artificial Sequence
  Synthetic Nucleotide sequence of the gapmer LNA-5′LASOalpha and of the gapmer LNA-ASOalpha antisense oligonucleotides
gcgcagtgattttttaaccatggga